(19) United States
(12) Patent Application Publication
(10) Pub. No.: US 2002/0077520 A1
(43) Pub. Date: Jun. 20, 2002

(54) Device and method for dilating and irradiating a vascular segment or body passageway

(76) Inventors: Jerome Segal, Chevy Chase, MD (US); Janet M. Hampikian, Decatur, GA (US); Neal A. Scott, Decatur, GA (US)

Correspondence Address: FINNEGAN, HENDERSON, FARABOW, GARRETT &, DUNNER LLP, 1300 I STREET, NW, WASHINGTON, DC, 20005, US

(21) Appl. No.: 09/735,239

(22) Filed: Dec. 13, 2000

Related U.S. Application Data
(63) Continuation-in-part of application No. 09/386,779, filed on Aug. 31, 1999.
(60) Provisional application No. 60/141,766, filed on Jun. 30, 1999.
(60) Provisional application No. 60/108,963, filed on Nov. 18, 1998.

(51) Int. Cl. A61N 5/00

(52) U.S. Cl. 600/1

(57) ABSTRACT

The present invention is directed to a device, and a method of using the device for dilating and irradiating a vascular segment, body passageway or an obstruction in a vascular segment or body passageway. The method of using the device comprises electroless deposition, electro-deposition or ion implantation of a radioactive coating on an expansion member. The radioactive coating may be deposited such that it has a total radioactivity that varies in at least one dimension of the expansion member. The expansion member may be substantially cylindrical and/or an expandable mesh. The radioactive expansion member, which is moveable between a radially contracted configuration and a radially expanded configuration, is radially contracted and placed into a vascular segment or body passageway. After advancing the contracted expansion member to a predetermined site in the vascular segment or body passageway, the expansion member is radially expanded to dilate and irradiate the predetermined site, while allowing fluid to flow through the expansion member. The expansion member is then radially contracted and removed from the vascular segment or body passageway.

This application is a continuation-in-part of application Ser. No. 09/386,779, filed Aug. 31, 1999, which claims the right of priority under 35 U.S.C. §119(e) to Provisional Applications No. 60/141,766, filed Jun. 30, 1999 and No. 60/108,963, filed Nov. 18, 1998.

### BACKGROUND OF THE INVENTION

1. Field of the Invention

The present invention relates to radioactive coating solutions, radioactive sols and sol-gels, methods used to form radioactive coatings on a variety of substrates, and to radioactive coated substrates. In particular, the present invention relates to a medical device, or a component thereof, having at least one radioactive coating layer thereon. The medical device is preferably an apparatus for dilating and irradiating an obstruction within a vascular segment or a body passageway, such as a catheter, more preferably a catheter utilizing a mesh for said dilating and irradiating functions.

2. Description of Related Art

Metal coatings are used in a variety of industrial and engineering applications to provide, for example, resistance to corrosion and wear, enhanced lubricity and decorative appearance. Several methods are used to form metal coatings, including electrodeposition and electroless deposition. Electrodeposition depends on the use of applied voltage to produce metal deposition, while electroless deposition depends on chemical reactions (including, the chemical reduction of a metal) independent of applied voltage. See, e.g., Dini, J. W., Developments and Trends in Electrodeposition, SAMPE Quarterly (1989) 28-32; and Ohno, I. Electrochemistry of Electroless Plating, Materials Science and Engineering, vol. A146 (1991) 33-49.

A wide variety of solutions for electrodeposition and electroless deposition are known, as theoretically any element or combination of elements, including metals and non-metals, can be added to a carrier metal to provide a suitable coating solution, wherein the carrier metal is present as an ion. In particular, metalloids including phosphorus and boron can be added to a carrier metal to provide a coating solution. Commonly used carrier metals include nickel, copper, cobalt, platinum, palladium, chromium, gold and silver. Particularly common are nickel and nickel alloy coating solutions, including nickel-phosphorus, nickel-boron, palladium-nickel, nickel-chromium, nickel-cobalt, nickel-phosphorus-boron, and copper-nickel chromium. Solutions are typically aqueous.

Electroless coatings are significantly more uniformly deposited than electrodeposited coatings, and are particularly desirable for coating complex shapes, including tubes and large components. Electroless deposition of nickel-phosphorus coatings, in particular, is well known. In general, electroless nickel phosphorus (ENP) coatings are dense, non-porous metal glass structures resembling polished stainless steel. ENP coatings typically contain between 3 and 13% by weight phosphorus, with the percentage significantly influencing both the chemical and physical properties of the coating. High phosphorus ENP coatings provide superior corrosion protection and are generally more continuous that lower phosphorus ENP coatings. R. P. Tracey, Practical Guide to Using N-P Electroless Nickel Coatings, Materials Selection and Design, 1990. ENP coatings are generally highly adhesive, providing resistance to chipping and peeling under extreme conditions. Electroless coatings may be amorphous or crystalline in structure.

Materials to be coated by electroless deposition are commonly metal. Electroless coatings can be applied to most metals and alloys, including steel and stainless steel, iron, aluminum, titanium, magnesium, copper, brass, bronze and nickel. In some cases, in addition to cleaning and removing surface oxides, the metal or alloy must be pre-treated to provide a catalytic surface for the electroless coating. For example, for coating Elgiloy™ with ENP, the surface must be coated (i.e., by electrodeposition or electroless deposition) with Ni prior to being coated with ENP. Electroless deposition may also be used to coat a variety of materials that are generally non-conductive, including plastics, glasses and ceramics, and composite materials. Coating of polymers generally requires additional steps to activate the polymer surfaces. A variety of processes are known for making polymer surfaces catalytic to the coating process. A tin-palladium catalyst, for example, can be absorbed onto the surface of the substrate, or applied as a catalytic coating.

Electroless deposition is carried out by immersing the substrate to be coated in a coating solution or bath comprising a carrier metal ion and a reducing agent. In ENP coating solutions, the most common reducing agent is hypophosphite ion (H₂PO₂). (Tracey, 1990). The metal ions are chemically reduced in the presence of the reducing agent and deposited onto the substrate surface. Deposition rates are typically 10-20 microns per hour. Typical commercial ENP coating are from about 2.5 to about 125 microns thick. (Tracey, 1990). Thicker coatings are typically required for rough surfaces.

Metal coatings may also be formed by electrodeposition. For example, nickel-phosphorous coatings may be produced by electrodeposition, and have comparable properties to those prepared via electroless deposition. Weil et al., Comparison of Some Mechanical and Corrosion Properties of Electroless and Electroplated Nickel-Phosphorous Alloys, Plating and Surface Finishing (Feb. 1989) 62-66.

Materials to be coated by electrodeposition include most metals and alloys, which in some cases must be clean and oxide free to provide a catalytic surface for electrodeposition. In certain circumstances, polymers may also be coated by electrodeposition. For example, plastics incorporating conductive particles can be coated by electrodeposition. Intrinsically conductive polymers may also be coated by electrodeposition. Generally, electrodeposition rates of Ni—P are higher than normally obtained via electroless methods. Also, electroplating solutions are more stable and have fewer replenishment problems. However, electrodeposited Ni—P does not coat complicated shapes with as uniform a thickness as ENP.

Electrodeposition is carried out by immersing the substrate to be coated in a coating solution or bath comprising a carrier metal ion and a radioisotope. Unlike electroless deposition, electrodeposition requires an applied current. In general, a reducing agent such as is necessary for electroless deposition is not required for electrodeposition, although reducing agents are not uncommonly present for eiectrodeposited Ni—P coatings, for example.

Methods for producing radioactive metal articles are also known. For example, it is known to manufacture a metal article comprising a radioisotope, e.g., by alloying the radioisotope with a metal or alloy or by ion implantation with a radioactive element. It is also known to manufacture non-radioactive metal articles which are subsequently made radioactive, e.g., by neutron bombardment. Each method of preparing radioactive metal articles, however, is associated with particular disadvantages. Manufacture of alloys using radioactive elements, for example, is problematic because many of the most desirable radioisotopes (e.g., P) show limited solubility as equilibrium alloying ingredients. Moreover, health physics safety issues associated with the manufacture of various articles effectively prohibit certain methods of manufacture.

The use of neutron bombardment to produce radioactive metal articles is similarly problematic, given limited access to nuclear reactors and tremendous costs. Neutron bombardment also constrains the size of components that can be irradiated. Moreover, neutron bombardment activates all components of the metal article that are susceptible to neutron activation, so that undesirable and potentially dangerous radioisotopes may be generated. Many standard alloy components, including Fe and Cr, form undesirable radiation reaction products. Thus, metals and alloys subject to neutron bombardment must be extremely pure and free of problematic elements, e.g., Na.

Coupled with the need to improve the method of depositing radioactive materials described above is the need to improve the delivery of radiation therapy within the human body. For example, cardiovascular disease is commonly accepted as being one of the most serious health risks facing our society today. Diseased and obstructed coronary arteries can restrict the flow of blood and cause tissue ischemia and necrosis. While the exact etiology of sclerotic cardiovascular disease is still in question, the treatment of narrowed coronary arteries is more defined. Surgical construction of coronary artery bypass grafts (CABG) is often the method of choice when there are several diseased segments in one or multiple arteries. Open-heart surgery is, of course, very traumatic for patients. In many cases, less traumatic, alternative methods are available for treating cardiovascular disease percutaneously. These alternate treatment methods generally employ various types of percutaneous transluminal angioplasty (PTCA) balloons or excising devices (atherectomy) to remodel or debulk diseased vascular segment segments. A further alternative treatment method involves percutaneous, intraluminal installation of expandable, tubular stents or prostheses in sclerotic lesions.

A recurrent problem with the previous devices and PTCA procedures is their failure to maintain patency due to the growth of injured vascular tissue. This is known as “restenosis” and may be a result of the original injury to the vessel wall occurring during the angioplasty procedure. Pathologically restenosis represents a neointimal proliferative response characterized by smooth muscle cell hyperplasia that results in reblockage of the vessel lumen necessitating repeat PTCA procedures up to 35-50% of all cases. It has been generally accepted that a radioisotope source may be capable of selectively inhibiting the growth of these hyperproliferating smooth muscle cells and thereby reduce the rate of restenosis after the primary interventional procedure.

Heretofore, various devices have been disclosed which may be used to expose a blood vessel undergoing angioplasty to intravascular radiation therapy. Balloon angioplasty catheters have been used to place and deploy a radioactive stent or prosthesis within human vessels. For example, in U.S. Pat. Nos. 5,059,166 and 5,176,617 a stent containing a radioactive source for irradiating an arterial segment to prevent restenosis is disclosed. In U.S. Pat. No. 5,199,939 an intravascular catheter and method for providing a radioactive means to the treated vessel segment is disclosed. In U.S. Pat. No. 5,616,114, an angioplasty balloon capable of inflation with a radioactive liquid for treatment of the affected vessel is described. U.S. Pat. No. 5,618,266 discloses a catheter for treating restenosis which contains a radioactive treatment source wire therein.

There are several disadvantages to using either a stent or balloon catheter to uniformly expose a vascular segment to radiation. Regarding the radioactive stent, once the stent is deployed, there is no means outside of invasive surgical excision, to remove the radioactive source from the vascular segment. Therefore, stents or implanted prostheses with radioactive properties must employ a radioisotope whose half-life and penetration properties must be precisely calibrated to deliver an exact quantity of radiation to the vascular segment upon stent deployment. Balloon catheters employed to irradiate a vascular segment have limitations including potential balloon rupture and ischemia due to the fact that balloons cannot be inflated within the vessel or vascular segment for long periods of time because it interrupts the flow of blood to distal vessels. This leads to tissue ischemia and potential necrosis. Even “perfusion” type angioplasty balloons used to deliver a radiation source to the affected artery provide far less than physiological blood flow during balloon inflation and dwell times are limited by ischemia and tissue necrosis. Simple intravascular catheters used to deliver alpha, beta or gamma radioactive source wire to the affected vessel do not permit centering of the radioactive source uniformly within the vessel lumen and therefore deliver radiation which is undesirably unequal to different walls of the vessel. Lack of centering the radiation source may provide up to four (4) times the radioactive dose to the vessel wall nearer the source than the wall farther from the source. Thus, it can be seen that there is a need not only for a new and improved device to selectively irradiate an arterial segment and which overcomes these disadvantages, but also for an improved method of rendering such a device radioactive.

It is one object of the present invention to provide a radioactive coating that can be produced from less than extremely pure materials, and without placing the coated article into a nuclear reactor.

It is a further object of the present invention to provide a radioactive coating comprising any of a wide variety of radioisotopes, including insoluble radioisotopes.

It is another object of the present invention to provide a radioactive coating solution which permits separation of the radioisotope therefrom.

It is yet another object of the present invention to provide a method of making a substrate radioactive by applying one or more radioactive coating layers thereto. It is another object of the present invention to provide radioactive coated substrates.

It is a further object of the present invention to provide substrates coated with multiple layers of radioactive coatings.

It is yet a further object of the present invention to provide a medical device, or a component of a medical device, coated with one or more radioactive coating layers.

It is a still further object of the present invention to provide a catheter having an component coated with one or more radioactive coatings layers, and more particularly, an expandable component coated with one or more radioactive coating layers.

It is yet a further object of the present invention to provide a mechanical dilatation device which is capable of dilating a vascular segment while providing radiation to the vascular segment segment.

It is yet a further object of the present invention to provide a mechanical dilatation device which is capable of dilating an obstruction within a vascular segment while providing radiation to the vascular segment segment.

It is yet a further object of the present invention to provide a percutaneous device which can be used for prolonged periods in exposing a vascular segment to an intravascular radiation source while allowing continuous perfusion of blood into and distal to the treatment area.

It is yet a further object of the present invention to provide a device that is not susceptible to structural damage (balloon rupture) and subsequent release of radioactive materials into the vasculature.

It is yet a further object of the present invention is to provide a device capable of providing a uniform dose of radiation to the vascular segment while dilating the vascular segment segment.

It is yet a further object of the present invention is to provide a device capable of providing a uniform dose of radiation to the vascular segment while dilating an obstruction within the vascular segment segment.

It is yet a further object of the present invention is to provide a device capable of placing the radioactive source directly in contact with the vessel wall in order to minimize distance to the target tissue.

It is yet a further object of the present invention is to provide a method of producing a mechanical dilatation device capable of dilating a vessel segment while providing radiation to the vessel segment.

It is yet a further object of the present invention is to provide a method of producing a mechanical dilatation device capable of dilating a vessel segment while providing radiation to the vessel segment and allowing continuous perfusion of blood into and through the treated vessel segment.

It is still a further object of the present invention to provide a method of making a substrate having a variable radioactive coating or coatings capable of producing an asymmetric radiation field.

It is yet a further object of the present invention to provide a substrate having a variable radioactive coating or coatings capable of producing an asymmetric radioactive field.

It is an object of the present invention to provide a brachytherapy device coated with a variable radioactive coating or coatings capable of producing an asymmetric radioactive field.

It is a further object of the present invention to provide a method of producing a radiation field corresponding to a target field.

It is a still further object of the present invention to provide a method of producing a radiation field corresponding to the morphology of a tumor.

### SUMMARY OF THE INVENTION

The present invention relates to radioactive coating solutions, radioactive sols and sol-gels, methods used to form a radioactive coatings on a substrate, and to radioactive coated substrates, particularly medical devices. It is known that radiation therapy can reduce the proliferation of rapidly growing cells. The present invention utilizes a radioisotope source with a mechanical dilatation device for enlarging a flow passage of a vessel or vascular segment by dilating and irradiating an obstruction in the vessel or vascular segment. Since the radioisotope source is capable of selectively inhibiting the growth of hyperproliferating cells, the present invention not only achieves acute patency of a vessel but employs radiation therapy to maintain chronic patency through the prevention of restenosis.

The present invention comprises a substantially cylindrically shaped expansion member and includes a means engaged to the expansion member for altering the distance between the proximal end and the distal end of the expansion member thereby transforming the expansion member between a diametrically contracted configuration and a diametrically expanded configuration. A radioisotope may be placed either inside the expansion member, alloyed into the metal from which the expansion member is constructed, coated onto the expansion member's exterior surface or alternately, the non-radioactive metal or alloy of the expansion member can be irradiated so that it becomes radioactive, i.e. it is then a radioisotope. The radioisotope can be an alpha, beta or gamma emitter, or any combination of these radiation sources.

The present method comprises the steps of advancing the radioactive expansion member or radioactive catheter to the obstruction in a vessel and applying opposed forces on said expansion member in an axial direction to move the expansion member to an expanded configuration wherein the expansion member dilates the vessel segment and the catheter/expansion member assembly irradiates the obstruction.

The present invention also relates to a method of making a substrate radioactive by applying a radioactive coating solution to the substrate to form a substrate having a radioactive coating formed thereon. To achieve the above-detailed benefits, the present invention relates to a coating solution comprising, in solution, at least one carrier metal ion and a radioisotope. In a particular embodiment of the present invention, the coating solution further comprises a reducing agent. The radioisotope present in the coating solution may be soluble or insoluble or present as the insoluble compound of a radioisotope.

In a particular embodiment of the method, the radioactive coating is a radioactive composite coating comprising a metal matrix and a radioactive dispersed phase. Methods of applying the radioactive coating solution to the substrate include electrodeposition and electroless deposition.

The present invention also relates a radioactive sols and radioactive sol-gels. The radioactive sol of the present invention comprises a metal alkoxide or other organometallic compound and a radioisotope. In a particular embodiment, the radioisotope is insoluble or the insoluble compound of a radioisotope, and is either added to the metal alkoxide or other organometallic compound prior to polymerization, or added by impregnation after partial polymerization. The present invention also relates to methods of making a substrate radioactive by applying a radioactive sol or sol-gel to a substrate to form a radioactive coating. In a particular embodiment of the present invention, the radioactive coating is a composite coating comprising an oxide matrix and a radioactive dispersed phase. Methods of applying the radioactive sol or sol-gel to the substrate include, without limitation, spin coating and dip coating.

The present invention further relates to methods of forming multiple radioactive coating layers on a substrate. Optionally, the method includes deposition of an activation layer over the substrate prior to deposition of the radioactive coating layer, such that the activation is interposed between the substrate and the radiation coating layer. In a particular embodiment, the method includes deposition of an activation layer between two radioactive coatings layers. Optionally, the method also includes deposition of a protective coating layer over the radioactive coating.

The present invention also relates to radioactively coated substrates. Suitable substrates include, but are not limited to, metals, alloys, polymers, plastics, ceramics and composites. In a particular embodiment of the present invention, the substrate is a medical device formed from such materials, or a component thereof. Representative medical devices include catheters, guidewires, stents, and brachytherapy devices. More particularly, the substrate is a catheter component, and more particularly, the expandable component of a catheter.

The present invention also relates to a method of making a substrate having a variable radioactive coating capable of producing an asymmetric radiation field, as well as to substrates having a variable radioactive coating. In addition to coating a mechanical dilatation device as described above, the present invention also relates to a brachytherapy device having a variable radioactive coating capable of producing an asymmetric radiation field.

The present invention advantageously permits production of radioactive substrates by virtue of a radioactive coating or coatings applied thereto. The present invention overcomes limitations of the traditional alloying and nuclear bombardment methods used to render metal articles radioactive to provide a radioactive metal coating which can be formed from a wide array of radioisotopes, including insoluble radioisotopes, relatively safely and inexpensively.

In particular embodiments, the present invention advantageously permits separation of a radioisotope from a radioactive coating bath, reducing the volume of the coating solution, which must be treated or disposed of as radioactive waste. This feature of the present invention also permits recharging of the radioisotope, providing a further economic benefit.

In other embodiments, the present invention advantageously permits production of a radioactive catheter including mesh component, sometimes referred to as a “radioactive dilatation/perfusion catheter” since it permits blood to flow through the mesh expansion member while in contact with the vessel wall. This allows increased dwell time without ischemia to the end organ, and thus the radioactivity of the catheter can be lowered to deliver the dose needed. For example, in the expanded state the radioactive source is placed against the inner vessel wall which is closest to the target tissue of the vessel (adventitia). Expansion of the mesh increases the vessel diameter and thus maximizes blood flow through the vessel while irradiating the target tissue. This in turn allows increased dwell time in the vessel without ischemia to the end organ

In certain instances, the expansion of the mesh can be used to relieve a blockage in a vessel at the same time as delivering the radiation. This obviates the need for relieving the vessel obstruction first and then applying the radiation. In other instances, the expansion of the mesh can be used to expand a stent in the vessel simultaneous with delivery of the radiation dose, thus obviating the need to separately place a stent within the vessel either prior to or following irradiation of the target segment.

The radioactive dilatation/perfusion catheters described above have mesh made of metal such as stainless steel or Elgiloy. The electrodeposited and electroless coatings described herein are suitable for such metal substrates, because such substrates conduct electricity, making it readily possible to employ them as the cathode in either process, electrodeposition or electroless deposition.

It would be advantageous to coat the woven metal mesh of a dilatation/perfusion catheter once the catheter is fully assembled thus obviating the need to assemble a catheter with radioactive parts. It is not desirable to place the assembled catheter into a reactor to activate the mesh since the entire catheter and all metallic structures therein would be rendered radioactive and since the material of the mesh (Cr, Co, Fe, Ni mixture) would have an undesirable half life and radiation emission.

The electroless plating and electroplating processes according to the present invention are thus ideal for rendering the mesh or distal bands present on the inner catheter member radioactive. Alloyed electrodeposited coatings made according to the present invention, and containing ³²P/P (such as Ni—³²P/P or Co—³²P/P, etc.) are particularly suitable for applying on a mesh catheter device as they are extremely robust coatings, as evidenced by their being adherent, wear and corrosion resistant, ductile, hard and smooth. Since the mesh of the radiation catheter device has wires that move and rub against each other when the mesh is diametrically contracted or expanded, these properties are advantageous for this application.

The above properties of the coatings, particularly excellent adherence of the coating to the underlying metal substrate, are beneficial for the following reasons. The catheter is activated inside a vessel or body cavity and it must be assured that the radioactive coating does not come off in the body. For example, once the mesh of the catheter is pressed up against the walls of a blood vessel it must be assured that the coating will remain in place even under pressures of 3-30 atm.

In addition, the coating must be ductile to withstand the activation (opening and closing) of the mesh which moves the elongate elements (wires) in relation to each other.

The coating is preferably uniform to provide a uniform radiation field and thin (on the order of 1-3 micron) in order to not increase the crossing profile of the catheter for crossing vessel blockages.

Alloyed coatings containing P, when applied under the appropriate conditions (proper surface pretreatment, solution chemistry, current density, temperature, etc.) are known to generally demonstrate most if not all of these properties.

In contrast, other forms of coatings, such as ceramic coatings, or polymeric coatings, may not exhibit the ductility necessary to remain adherent to the device without delamination. Ceramic materials in general are very brittle, and the flexing of a mesh catheter as it diametrically contracts and expands would cause fracture and delamination of ceramic coatings. Many polymeric materials are prone to radiation embrittlement, and thus radioactive polymeric coatings may also prove problematic.

Further, a fully assembled mesh catheter device is able to withstand the highly acidic electroplating solution. Other devices may not be able to be coated in their fully assembled state, which would result in a requirement for the (dangerous) hand assembly of a device from radioactive materials.

Finally, the application of a coating, for example, of NiP to a mesh catheter device actually improves the overall smoothness of the surface of the mesh component of the catheter. This results in a mesh that is less likely to “stick” to the artery wall and cause trauma to the vessel wall.

In addition to the above advantages, the mesh component of the dilatation/perfusion catheter device does not contain significant trace ingredients such as Cu that might “poison” the bath (by dissolution) and prevent it from plating with the target properties (in particular, smoothness).

Also, an extremely uniform (in composition and thickness) coating can be produced on the dilatation/perfusion device, when deposited using a solution vessel that is cylindrical, containing a cylindrical anode equidistant from the catheter. This uniform coating in turn causes the catheter to produce a radioactive field that is also uniform. This inventive method contrasts with other coating methods such as those involving dipping of the part into a solution and subsequent drying by spinning or other methods geared toward leaving a uniform thickness coating on the part.

These and other advantages of the present invention will be apparent to those skilled in the art in view of the disclosure set forth below.

### BRIEF DESCRIPTION OF THE FIGURES

FIG. 1 is a side-elevation view partially in section of a mechanical dilatation and irradiation device incorporating the present invention.

FIG. 2 is a cross-sectional view taken along the line 2-2 of FIG. 1.

FIG. 3 is a cross-sectional view taken along the line 3-3 of FIG. 1.

FIG. 4 is a cross-sectional view taken along the line 4-4 of FIG. 1.

FIG. 5 is a cross-sectional view taken along the line 5-5 of FIG. 1.

FIG. 6 is a cross-sectional view taken along the line 6-6 of FIG. 1.

FIG. 7 is a greatly enlarged view of a portion of the dilatation and irradiation device in a partially expanded state.

FIG. 8 is a partial side-elevation view of another embodiment of a mechanical dilatation and irradiation device incorporating the present invention with a part of the device covered by a protective material to prevent damage to the vessel wall.

FIG. 9 is a partial side-elevation view of another embodiment of a mechanical dilatation and irradiation device incorporating the present invention which can be utilized in conjunction with a rapid exchange technique.

FIG. 9a is an enlarged side-elevation view of the rapid exchanged embodiment of the mechanical dilatation and irradiation device demonstrating the guidewire entry ports in the inner and outer elongated tubular members.

FIG. 10 is a side-elevation view partially in section of a mechanical dilatation and irradiation device incorporating another embodiment of the present invention.

FIG. 11 is an enlarged cross-sectional view taken along the line 11-11 of FIG. 10.

FIG. 12 is an enlarged cross-sectional view taken along the line 12-12 of FIG. 10.

FIG. 13 is an enlarged side-elevation view of a portion of the device shown in FIG. 10 looking along the line 13-13.

FIG. 14 is a cross-sectional view taken along the line 14-14 of FIG. 13.

FIG. 15 is a cross-sectional view taken along the line 15-15 of FIG. 12.

FIG. 16 is a cross-sectional view similar to FIG. 15 but showing the use of a braid rather than a coil spring.

FIG. 17 is a greatly enlarged fragmentary view taken along the line 17-17 of FIG. 13.

FIG. 18 is a side-elevation view of the distal extremity of the device shown in FIGS. 10-14 showing the distal extremity with the expansion member in an expanded condition.

FIG. 19 is a side-elevation view of the expandable mesh with a series of bands on the inner tubular member that are radioactive.

FIG. 20 is a cross sectional view of the radiated flexible elongated elements demonstrating the emission of radiation into the blood vessel.

FIG. 21 is a cross sectional view demonstrating the symmetrical emission of radiation from the inner tubular member located within the expandable mesh.

FIG. 22 is a cross sectional view of the one flexible elongate element (wire) of the expandable mesh demonstrating the symmetrical emission of radiation from the elongate element fabricated with a material which alloys or incorporates the radioisotope within the material.

FIG. 23 is a cross sectional view of the one flexible elongate element (wire) of the expandable mesh demonstrating the symmetrical emission of radiation from a radioactive solid or liquid core within the elongate element.

FIG. 24 is a cross sectional view of the one flexible elongate element (wire) of the expandable mesh demonstrating the symmetrical emission of radiation from a radioactive coating over the elongate element.

FIG. 25 is a cross sectional view of the mechanical dilatation and irradiation device deployed within an arterial segment demonstrating the symmetrical emission of radiation.

FIG. 26 is an isodensity curve of the radioactive coating applied to a catheter according to Example 1, as measured along the catheter's long axis, illustrating uniformity of deposition.

FIG. 27 is an isodensity curve of the radioactive coating applied to a catheter according to Example 1, as measured along the catheter's short axis, illustrating uniformity of deposition.

FIG. 28 is a Ni-25 at %P electroless coating deposited onto Elgiloy™ in sheet form, viewed in cross-section via scanning electron microscopy (SEM).

FIG. 29 is a coated and uncoated Full Flow catheter component by SEM images.

FIG. 29A depicts a Full-Flow device coated with a Ni-26 at %P electroless coating. The coating is approximately 7 microns thick, and is uniform in appearance.

FIG. 29B depicts an uncoated Full-Flow device.

FIG. 30 is a cross-section of the Full-Flow device of FIG. 29A.

FIG. 30A depicts SEM at 100×.

FIG. 30B depicts SEM at 300×.

FIG. 31 is an energy dispersive x-ray spectrum from a Ni—P electroless electroless coating, showing Ni and P peaks, corresponding quantitative analysis indicates concentration of coating being, about 26 mol or atomic %P (or about 15.8 wt. % P).

FIGS. 32A and 32B are x-ray diffraction spectrums showing, respectively, the uncoated Elgiloy™ and the Ni—P electrolessly coated Elgiloy™ of FIG. 29. The uncoated alloy (32A) shows crystalline peaks consistent with the substrate; the coated alloy (32B) shows a diffuse peak consistent with the coating being amorphous as expected for a high phosphorus coating.

FIG. 33 depicts substrates having a radioactive coating or coatings formed thereon. FIG. 33A depicts a substrate having an electroless radioactive coating. FIG. 33B depicts a substrate comprising multiple radioactive coating layers.

### DETAILED DESCRIPTION OF SPECIFIC EMBODIMENTS

The invention disclosed herein relates to radioactive coating solutions, radioactive sols and sol-gels, methods used to form a radioactive coatings on a substrate, and to radioactive coated substrates, such as medical devices used to dilate and irradiate a segment of a blood vessel.

A device according to the present invention is comprised of an expansion member to be disposed in an obstruction in a vessel carrying flowing blood. The expansion member has first and second ends and an intermediate portion between the first and second ends. The expansion member also has a flow passage extending therethrough with a diameter and a longitudinal central axis. The diameter of the flow passage is a variable with movement of the first and second ends relative to each other along the longitudinal central axis from a diametrically contracted position to a diametrically expanded condition. The cylindrical expansion member is comprised of a plurality of flexible elongate elements, each of which extends helically about the longitudinal extending central axis. In one embodiment of the present invention, a radiation source is located within the distal end of the device comprising a plurality of bands secured to the central axis that are either alloyed with a radioactive material or fabricated with a material that can subsequently become radioactive.

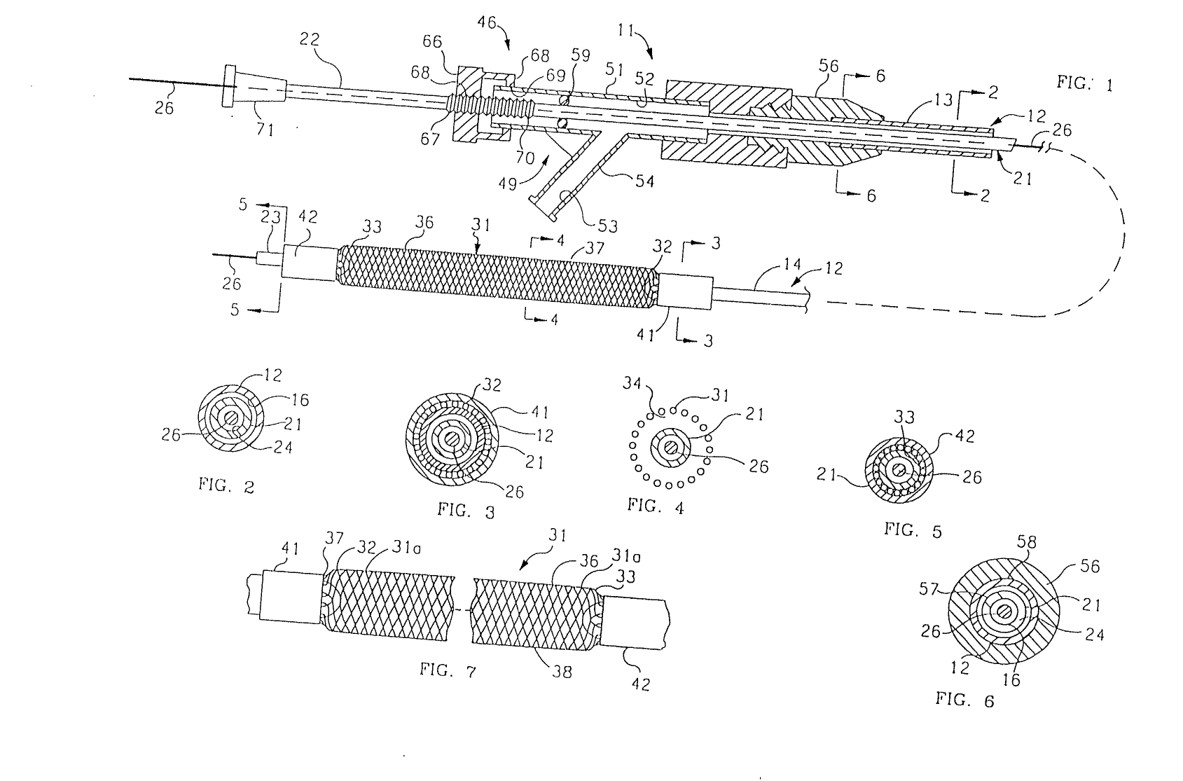

In another embodiment, the flexible elongate elements are either alloyed with a radioactive material or fabricated with a material that can subsequently become radioactive. A plurality of the flexible elongate elements having a first common direction of rotation are axially displaced relative to each other and cross a further plurality of the flexible elongate elements also axially displaced relative to each other but having a second common direction opposite to that of the first direction of rotation to form a braided cylindrical expansion member. The crossing of the flexible elongate elements occurs in an area of contact between the flexible elongate elements. First and second means is provided respectively engaging the first and second ends of said cylindrical expansion member for retaining said first and second ends in contracted positions. Mechanisms are provided for causing relative axial movement of the first and second ends towards each other to cause the intermediate cylindrical portion of the expansion member to contact longitudinally and to expand diametrically by causing the flexible elongate elements in the intermediate portion of the cylindrical member to move closer to each other expanding the diametric dimensions of the cylindrical expansion member. Flexible elongate elements at the first and second ends of the cylindrical expansion member remain contracted around and within first and second means and are thereby prevented from moving closer which maintains spacing between the flexible elongate members so that blood in the vessel can continue to flow through the first and second ends and through the flow passage in the cylindrical expansion member while the cylindrical expansion member is in its diametrically expanded state and in engagement with the vessel walls or obstruction. As shown in FIGS. 1-7 of the drawings, which show the mechanical dilatation and irradiation device 11 shown therein consists of a first or outer flexible elongate tubular member 12 having proximal and distal extremities and 14 with the flow passage 16 extending from the proximal extremity 13 to the distal extremity 14. A second or inner flexible tubular member 21 is coaxially and slidably disposed within the flow passage 16 of the first or outer flexible elongate tubular member 12 and is provided with proximal and distal extremities 22 and 23 with a flow passage 24 extending from the proximal extremity 22 to the distal extremity 23.

A guide wire 26 of a conventional type is adapted to be introduced through the flow passage 24 in the inner flexible elongate tubular member for use in guiding the mechanical dilatation and irradiation device 11 as hereinafter described. The guide wire 26 can be of a suitable size as for example 0.010″-0.035″ and can have a suitable length ranging from 150 to 300 centimeters. For example, the first or outer flexible elongate tubular member 12 can have an outside diameter of 0.6-3 millimeters with a wall thickness of 0.12 millimeters to provide a flow passage of 0.75 millimeters in diameter. Similarly, the second or inner flexible elongate tubular member 21 can have a suitable outside diameter as for example 0.6 millimeters with a wall thickness of 0.12 millimeters and a flow passage 24 of 0.45 millimeters in diameter. The flexible elongate tubular members 12 and 21 can be formed of a suitable plastic as for example a polyimide, polyethylene, Nylon or polybutylterphalate (PBT).

In accordance with the present invention an expansion member 31 is provided which has a first or proximal end 32 and a second or distal end 33 with a central or inner flow passage 34 extending from the proximal end 32 to the distal end 33 along a longitudinally extending central axis and has a diameter which is a variable as hereinafter described. The expansion member 31 is comprised of a plurality of flexible elongate elements or filaments 36, each of which extends helically about the longitudinally extending central axis. The flexible elongate elements 36 are formed of suitable materials which can be utilized in the human blood as for example stainless steel, Nitinol, Aermet™, Elgiloy™ or certain other plastic fibers. The flexible elongate elements 36 can have a suitable diameter as for example 0.001 to 0.010 inches or can be configured as a round, elliptical, flat or triangular wire ribbon. A plurality of the flexible elongate elements 36 have a first common direction of rotation about the central axis as shown in FIGS. 1, 7, 8 and 15 are axially displaced relative to each other and cross a further plurality of the flexible elongate elements 36 also axially displaced relative to each other but having a second common direction of rotation opposite to that of the first direction of rotation to form a double helix or braided or mesh-like cylindrical expansion member with the crossing of flexible elongate elements 36 occurring in the area of contact between the flexible elongate elements to form openings or interstices 37 therebetween. Thus the flexible elongate elements 36 form an expansion member which provides a central or inner flow passage 34 which is variable in diameter upon movement of the first and second ends of the expansion member 31 relative to each other along the longitudinally extending central axis. Means is provided for constraining the first and second or proximal and distal ends 32 and 33 of the expansion member 31 and consists of a first or proximal collar 41 and a second or distal collar 42. The first and second collars 41 and 42 are formed of a suitable material such as a polyimide. The first or proximal collar 41 has a suitable length as for example 1.0 to 5.0 millimeters and is sized so that it can fit over the first or proximal end 32 of the expansion member when it is in a contracted position and over the distal extremity 14 of the first or outer flexible elongate member 12. In order to ensure that elongate elements or filaments 36 of the first or proximal extremity 32 are firmly secured to the distal extremity 14 of the first or outer flexible elongate member 12, an adhesive can be provided bonding the first or proximal end 32 to the collar 41 and to the distal extremity 14 of the first or outer flexible elongate tubular member 12. The second or distal collar 42 can be of a suitable size and typically may be slightly smaller in diameter because it need merely secure the elongate element or filaments 36 of the distal end 33 of the expansion member 31 to the distal extremity 23 of the second or inner flexible elongate tubular member 21. An adhesive (not shown) is provided to firmly secure the second or distal end 33 of the expansion member 31 between the second or distal collar 42 and the distal extremity of the inner flexible elongate tubular member 21. In this manner it can be seen that the cylindrical expansion member 31 has its proximal end curved conically inward toward and secured to the distal extremity of the outer flexible elongate tubular member 12 and the second or distal end 33 of the expansion member 31 also curves conically inward toward and is secured to the distal extremity of the second or inner flexible elongate tubular member 21.

Typically the distance between the first and second collars 41 and 42 can range from between 5 to 150 millimeters. Typically the distal end 23 of the second or inner flexible elongate tubular member 21 extends approximately 5-170 millimeters beyond the distal extremity 14 of the first or outer flexible elongate tubular member 12.

It can be seen that by moving the first or outer flexible elongate tubular member 12 and the second inner flexible elongate tubular member 21 axially with respect to each other, the first and second ends of the expansion member 31 are moved towards each other causing the elongate elements or filaments 36 of an intermediate portion of the cylindrical expansion member between the first and second ends to move closer to each other to cause these flexible elongate elements to move into apposition with each other and to expand in a first radial direction the intermediate portion of the cylindrical expansion member 31 (FIG. 7) and to cause the diameter of the central flow passage to increase. The portions of the expansion member 31 immediately adjacent the first and second collars 41 and 42 remain restrained by the collars 41 and 42 causing the flexible elongate elements 36 immediately adjacent to the collars 41 and 42 to curve conically toward and remain crossed and unable to come into close apposition and thereby provide openings or interstices 37 therebetween, which remain relatively constant in shape and size so that blood can flow from the first and second ends 32 and through the central or inner flow passage 34 as hereinafter described.

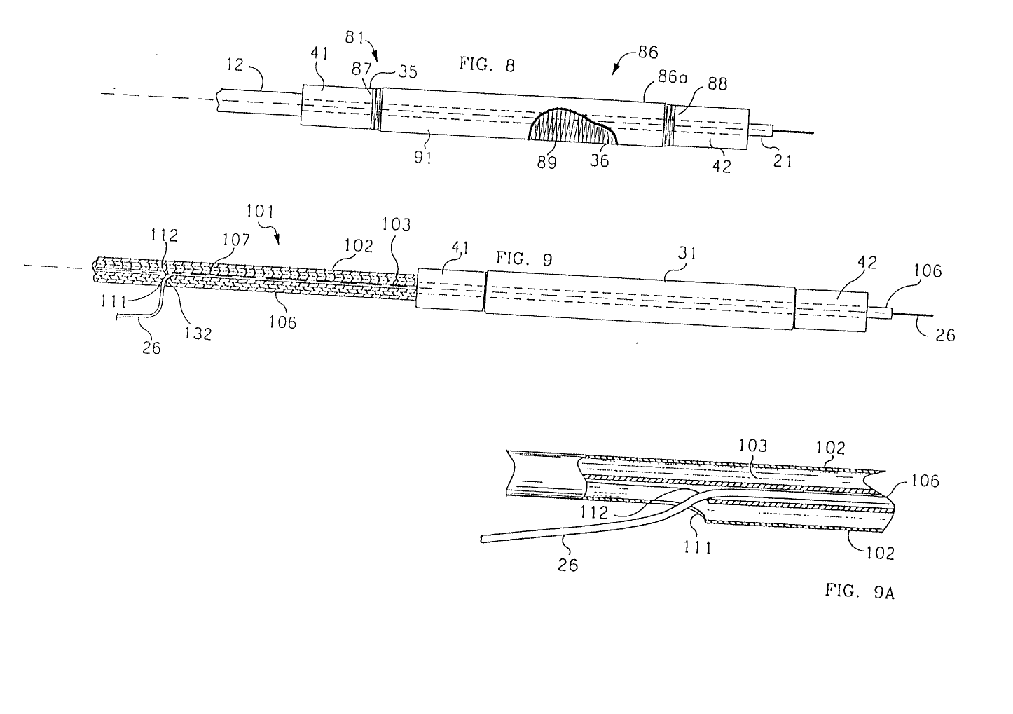

Mechanisms are provided in the mechanical dilatation and irradiation device 11 for causing relative movement between the first or outer flexible elongate tubular member 12 and the second or inner flexible elongate tubular member 21 and consists of a screw mechanism 46. The screw mechanism 46 includes a Y-adapter 49 which is provided with a central arm 51 having a lumen 52 through which the second or inner flexible elongate tubular member 21 extends. The lumen or flow passage 52 is in communication with the lumen 16 of outer flexible elongate tubular member 12 and with a flow passage 53 in a side arm 54 which is adapted to receive a syringe (not shown) so that saline, radiocontrast liquid or a drug can be introduced through the side arm 54 and into the flow passage 52 in the Y-adapter 49 and thence into lumen 16 of outer member 12. The distal end of screw mechanism 46 is provided with a fitting 56 with inner lumen 57 (see FIG. 6) into which the proximal end 13 of flexible elongate tubular member 12 is seated and held in place by an adhesive 58 at the distal end of fitting 56. Lumen 57 is thereby in communication with flow passage 52 of central arm 51 and with flow passage 53 of side arm 54. An O-ring 59, which is adapted to form a fluid-tight seal with respect to the second or inner flexible tubular member 21, is disposed in the lumen of the central arm 51. An interiorly threaded knurled knob 66 is threaded onto an exteriorly threaded member 67 which is secured to and surrounds the proximal extremity 22 of inner flexible elongate tubular member 21. The knob 66 is provided with an inwardly extending flange 68 which seats in an annular recess 69 in the central arm 51. Thus, rotation of the knob 66 causes advancement or retraction of threaded member 67 and the second or inner flexible elongate tubular member 21 with respect to the fitting 56. Indicia 68 in the form of longitudinally spaced-apart rings 70 are provided on the member 67 and serve to indicate the distance which the second or inner flexible elongate tubular member 21 has been advanced and retracted with respect to the first or outer flexible elongate member 12.

A Luer-type fitting 71 is mounted on the proximal extremity 22 of the inner elongate flexible tubular member 21 and is adapted to be engaged by a finger of the hand. The guide wire 26 extends through the fitting 71 and into the lumen 24 of inner elongate flexible tubular member 21.

It should be appreciated that even though one particular screw mechanism 46 has been provided for advancing and retracting the flexible elongate members 12 and with respect to each other, other mechanisms also can be utilized if desired to provide such relative movement. Other possible designs that could be employed are scissors-jack, rachet-type or straight slide mechanisms.

In order to provide the desired radiopacity for the distal extremity of the mechanical dilatation and irradiation device 11 so that it can be observed fluoroscopically during a dilatation procedure, the collars 41 and 42 can be formed of a radiopaque material as for example by filling the polymeric material with radiopaque particles of a suitable material such as barium or by providing collars containing radiopaque metals, such as tungsten or platinum or a tungsten/platinum alloy. Although the flexible elongate elements 36 which comprise the expansion member 31 have some radiopacity by being formed of a stainless steel or other suitable material such as Elgiloy, there normally is insufficient radiopacity for most medical procedures. Therefore to augment the radiopacity of the expansion member 31, radiopaque wire of a suitable material such as platinum or tungsten can be wound along with the flexible elongate element 36 to provide the necessary radiopacity. This often may be desirable because this would make it possible to ascertain the position of the cylindrical expansion member and its diameter as it is expanded and retracted between a minimum contracted position and a maximum expanded position by relative movement between the distal extremities of the first or outer flexible elongate member 12 and the second or inner flexible elongate tubular member 21. The use of the helical wraps of platinum does not significantly interfere with the general mechanical properties of the expansion member 31 desired in connection with the present invention. Alternatively, the flexible elongate elements 36 may be plated with a radiopaque metal such as platinum or gold to enhance their radiopacity. Alternatively, the flexible elongate elements may be comprised of hollow wires, the central core of which may be filled with radipaque metals such as tungsten, gold or platinum or with compound salts of high radiopacity.

To perform as a radioactive source for the present invention, the flexible elongate elements themselves can be radioactive as described in more detail below. The flexible elongate elements may be alloyed with a material, coated with a material, or have a central lumen that can be filled with a material, that is radioactive or has been made radioactive utilizing one of the activation mechanisms known by those skilled in the art.

Operation and use of the mechanical dilatation and irradiation device 11 may now be briefly described as follows. Let it be assumed that the patient upon which the medical procedure is to be performed utilizing the mechanical dilatation and irradiation device 11 has one or more stenoses which at least partially occlude one or more arterial vessels supplying blood to the heart and that it is desired to enlarge the flow passages through these stenoses. Typically the mechanical dilatation and irradiation device 11 would be supplied by the manufacturer with the cylindrical expansion member 31 in its most contracted position to provide the lowest possible configuration in terms of diameter and so that the diameter approximates the diameter of the outer flexible elongate tubular member 12. Thus, preferably, it should have a diameter which is only slightly greater than the tubular member 12, as for example by 1.0-2.3 millimeters. The first and second collars 41 and 42 also have been sized so they only have a diameter which is slightly greater than the outer diameter of the outer flexible elongate tubular member 12. To bring the cylindrical expansion member 31 to its lowest configuration, the screw mechanism 46 has been adjusted so that there is a maximum spacing between the distal extremity 23 of the inner flexible elongate tubular member and the distal extremity 14 of the outer flexible elongate tubular member 12. In this position of the expansion member 31, the flexible elongate elements 36 cross each other at nearly right angles so that the interstices or openings 37 therebetween are elongated with respect to the longitudinal axis.

The mechanical dilatation and irradiation device 11 is then inserted into a guiding catheter (not shown) typically used in such a procedure and introduced into the femoral artery and having its distal extremity in engagement with the ostium of the selected coronary artery. Thereafter, the guide wire 26 can be inserted independently of the mechanical dilatation and irradiation device 11. If desired the guide wire 26 can be inserted along with the mechanical dilatation and irradiation device 11 with its distal extremity extending beyond the distal extremity of device 11. The guide wire 26 is then advanced in a conventional manner by the physician undertaking the procedure and is advanced into the vessel containing or having contained a stenosis. The progress of the distal extremity of the guide wire 26 is observed fluoroscopically and is advanced until its distal extremity extends distally of vessel segment or the stenosis. With the expansion member 31 in its diametrically contracted position and the prosthesis secured thereon, the mechanical dilatation and irradiation device 11 is advanced over the guide wire 26. The distal extremity 23 of the second or inner flexible elongate tubular member 21 is advanced through the stenosis over the guide wire 26 until it is distal to the vessel segment or the stenosis and so that the distal extremity 14 of the first or outer flexible elongate tubular member 12 is just proximal of the vessel segment or the stenosis.

After the expansion member 31 is in a desired position in the vessel segment or the stenosis, the expansion member 31 is expanded from its diametrically contracted position to an expanded position by moving the distal extremities 14 and 23 closer to each other by operation of the screw mechanism 46. This can be accomplished by holding one distal extremity stationary and moving the other distal extremity towards it or by moving both distal extremities closer to each other simultaneously. This movement of the distal extremities 14 and 23 causes collars 41 and 42 to move closer to each other and to cause the central flexible elongate elements 36 forming the double helix mesh of the intermediate portion 31 a of the flexible cylindrical expansion member 31 to move relative to each other to progressively decrease the vertical crossing angle of the double helically wound flexible elongate elements 36 from approximately 140° to 170° in its extended state to 5° to 20° in its axially contracted state and to progressively change the interstices or openings 37 from diamond-shaped openings with long axes parallel to the central longitudinal axis of the catheter in its extended state to substantially square-shaped openings in its intermediately contracted state to elongate diamond-shaped interstices or openings with the longitudinal axes extending in directions perpendicular to the central longitudinal axis with the flexible elongate elements 36 coming into close apposition to each other while at the same time causing radial expansion of the expansion member and to progressively increase the diameter of the central flow passage 34. The enlargement of expansion member 31 in addition to being viewed fluoroscopically can also be ascertained by the indicia 68 carried by the threaded member 67.

During the time that the expansion member 31 is being expanded, it exerts radial forces against the vessel wall or alternately a stent, thereby expanding the vessel wall or stent against the vessel wall or stenosis. If employed, the stent compresses against and becomes implanted within the wall of the vessel thereby enlarging the vessel lumen or the stenosis so that an increased amount of blood can flow through the vessel. The intermediate portion 31 a of the expansion member 31 when fully expanded is almost a solid tubular mass which has significant radial strength to fully expand the vessel lumen, stent or prosthesis. In addition, because of spring-like properties of the enlarged expansion member being comprised of helically wound flexible elongate elements 36, the expansion member 31 can conform to a curve within the blood vessel while still exerting significant radial force to the vessel wall, stent or prosthesis and to make possible expansion of the vessel lumen or compression of the stenosis without tending to straighten the curve in the vessel which typically occurs with standard straight angioplasty balloon systems. Since the expansion member can be comprised of flexible elongate elements that themselves are a radiation source (see FIG. 22), or alternatively have a hollow core containing a radiation source (see FIG. 23), or alternatively are coated or alloyed with a radiation source, uniform alpha, beta, or gamma radiation can be delivered to the vessel during the time of device expansion (see FIGS. 20, 25).

Since the ends of the expansion member 31 are constrained by the proximal and distal collars 41 and 42, the flexible elongate elements 36 form a braided mesh of the expansion member 31 adjacent to the distal extremity 23 of the inner elongate flexible tubular member 21 and the distal extremity 14 of the outer flexible elongate tubular member 12 under the collars 41 and 42, respectively, are held in substantially constant angular relationship to each other with the vertical crossing angles between 5° and 170° and are unable to come into close apposition with each other. Therefore the interstices or openings 37 adjacent the collars 41 and 42 remain open because the flexible elongate elements 36 are unable to change from their relatively fixed crossed positions. Blood continues to flow through the central or inner flow passage 34 by passing through the openings 37 in the first or proximal end 32 into the central or inner passage 34 and out the openings in the second or distal end 33. Thus, blood flow through the vessel is not impeded by the expansion of the expansion member 31. Since blood flows continuously through the dilatation and irradiation device during the dilatation and irradiation procedure, there is minimal danger of ischemia occurring. This makes it possible to maintain dilatation and irradiation of the obstruction over extended periods of time when desired. One particularly advantage for the mechanical dilatation and irradiation device 11 is that it could be used with patients which have obstructions of a critical nature that cannot even tolerate relatively short periods of balloon dilatation without leading to ischemia creating permanent damage or shock to the patient. Another advantage of the present invention is that uniform exposure of radiation to the vessel wall can be accomplished during this time.

The open construction of the expansion member 31 also serves to prevent blocking off of other vessels branching off from the vessel in the region in which dilatation and irradiation procedures are being performed because the blood can flow through the central interstices 38 of the expansion member 31.

After dilatation and irradiation of the lesion has been carried out for an appropriate length of time, the expansion member 31 can be moved from its expanded position to a contracted position by operation of the screw mechanism 46 in a reverse direction to cause separation of the distal extremities 14 and 23 to thereby cause elongation of the expansion member 31 with a concurrent reduction in diameter.

After the expansion member 31 has been reduced to its contracted or minimum diameter, the mechanical dilatation and irradiation device 11 can be removed along with the guide wire 26 after which the guiding catheter (not shown) can be removed and the puncture site leading to the femoral artery closed in a conventional manner. Although, the procedure hereinbefore described was for treatment of a single stenosis, it should be appreciated that if desired during the same time that the mechanical dilatation and irradiation device 11 is within the guiding catheter, other vessels of the patient having stenoses therein can be treated in a similar manner merely by retracting the distal extremity of the mechanical dilatation and irradiation device 11 from the stenosis being treated and then advancing it into another stenosis in another vessel in a similar manner.

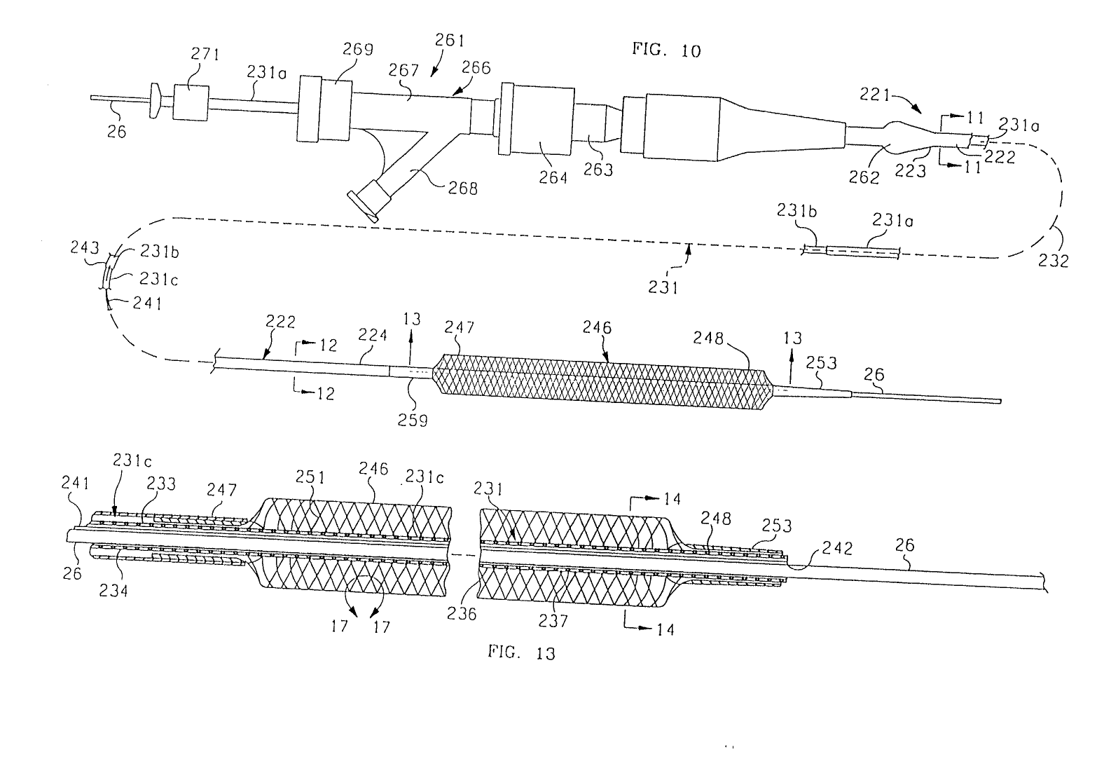

Another embodiment of a dilatation and irradiation device incorporating the present invention is shown in FIGS. 9 and 9a. As shown therein, the mechanical dilatation and irradiation device 101 is constructed in a manner similar to the mechanical dilatation and irradiation device 11 with the exception that it is provided with rapid exchange capabilities. This is accomplished by providing an outer flexible elongate tubular member 102 having a lumen 103 therein and an inner flexible elongate tubular member 106 having a lumen 107 which have the expansion member 31 secured thereto by the proximal and distal collars 41 and 42. The outer flexible elongate tubular member 102 is provided with a port or opening 111 into the corresponding lumen 103 and which is 13-60 centimeters from the distal extremity 32 of the expansion member 31. A corresponding port or opening 112 into corresponding lumen 107 is provided within the inner flexible elongate tubular member 106. These ports 111 and 112 are positioned so that when the expansion member 31 is in its expanded position with the distal extremities of the members 102 and 106 being in closest proximity to each other, the openings 111 and 112 are in registration with each other. In this position, the mechanical dilatation and irradiation device 101 can be loaded onto the guide wire 16 by advancing the most proximal extremity of guide wire 26 first into lumen 107 of the distal extremity of the inner flexible elongate member 106 and then back through port or opening 112 and port 111 which are in registration and out of the flexible elongate tubular member 102. The expansion member 31 is next contracted from its diametrically expanded condition to a contracted condition by moving the distal extremities of outer and inner flexible elongate tubular members 102 and 106 further apart by operation of screw mechanism 46. This procedure is performed while maintaining a stable position of the external position of guide wire 26 in a constant position in relation to port 111. As the distal extremity of flexible tubular member 106 is moved further from the distal extremity of flexible elongate tubular member 102, port 112 will move out of registration with port 111 while maintaining guide wire 26 within lumen 107 and advancing the distal extremity of the flexible elongate tubular member 106 along the guide wire 26. In this diametrically contracted state of the expansion member 31, the mechanical dilatation and irradiation device 101 may be advanced along guide wire 26 through the region of stenosis in the blood vessel and enlargement of expansion member 31 may occur using screw mechanism 46 in the manner previously described. Once dilatation and irradiation has been completed, expansion member 31 can be diametrically contracted and the mechanical dilatation and irradiation device 101 may be removed from the blood vessel and the guiding catheter by maintaining a stable position of guide wire 26 in relation to the blood vessel and retracting device 101 along guide wire 26 until the distal extremity of inner flexible member 106 exits the patient's body. The mechanical dilatation and irradiation device 101 may now be rapidly exchanged with another mechanical device 101 as for example, one having an expansion member 31 which can be increased to a larger diameter over a standard 175 to 185 centimeter length guide wire 26.

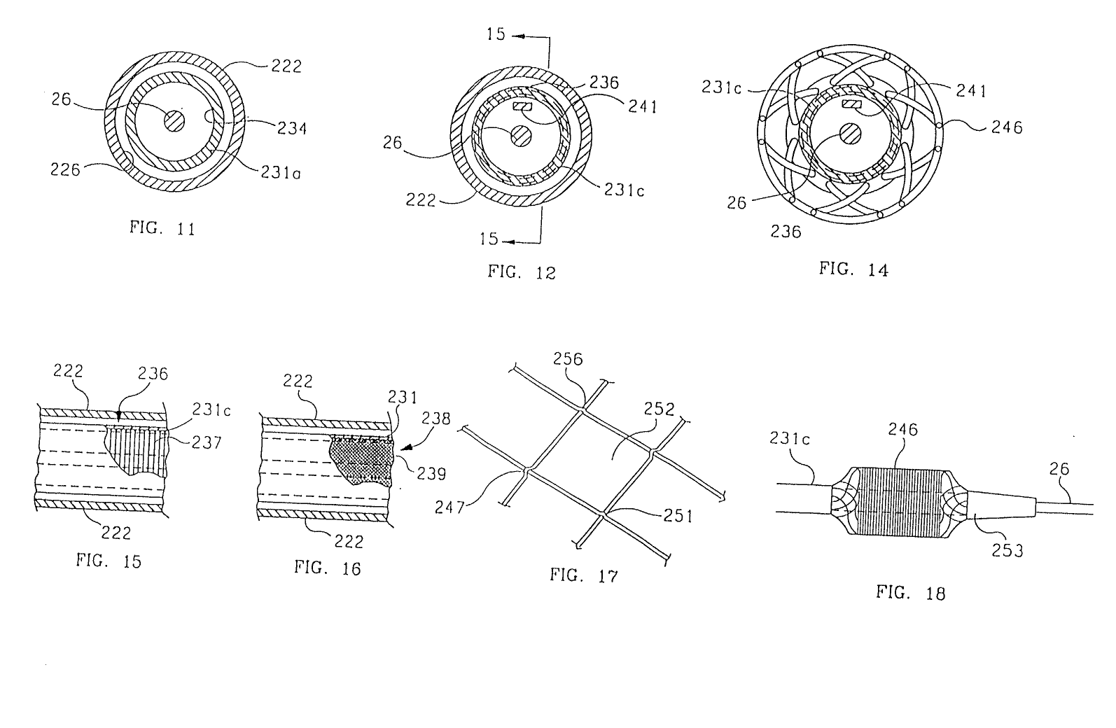

Another embodiment of a mechanical dilatation and irradiation device 221 incorporating the present invention is shown in FIGS. 10-16. As shown therein, the device 221 consists of a flexible elongate tubular member 222 having proximal and distal extremities 223 and 224. The flexible elongate tubular member 222 can be formed out of a suitable material such as a polyethylene or a polyimide.

A lumen 226 extends from the proximal extremity 223 to the distal extremity 224 and has a size which is the same as in the first or outer flexible elongate tubular member 12 hereinbefore described in connection with the previous embodiments. Thus, it can have a suitable size as for example 3-5 French. A second or inner flexible elongate tubular member 231 is provided which is slidably and coaxially disposed within the lumen 226. It is provided with proximal and distal extremities 232 and 233 with a lumen 234 extending from the proximal extremity 232 to the distal extremity 233. In the present embodiment of the invention, the inner flexible elongate tubular member 231 serves as a support member. The flexible elongate tubular member 231 is formed of three portions 231a, 231b and 231c with the first portion 231a being at the proximal extremity 232 and the second portion 231b extending from the proximal extremity 232 to the near distal extremity 233. The portion 231a is formed of a hypotube having an outside diameter of 0.010″ to 0.042″ and an inside diameter of 0.012″ to 0.030″ to provide a wall thickness of 0.002″ to 0.010″. The portion 231a has a suitable length as for example 10-30 centimeters. The second portion 231b can be formed so that it has an outside diameter of 0.016″ to 0.042″ and an inside diameter of 0.012″ to 0.030″ to provide a wall thickness of 0.002″ to 0.010″. Thus it can be seen that the portion 231a has a greater wall thickness and provides additional stiffness and rigidity. A guide wire of the type hereinbefore described is slidably disposed in the lumen 234. The lumen 234 in the flexible elongate tubular support member 231 is sized so that it can readily accommodate the guide wire 26. Thus, if a guide wire having a size 0.014″ is used, the lumen 226 should have a diameter which is greater than 0.016″ to 0.018″.

The third portion 231c of the flexible elongate tubular support member 231 is formed of a suitable material such as plastic, as for example a polyimide. It has a suitable length, as for example from 20-40 centimeters and preferably a length of approximately 30 centimeters. The portion 231c is bonded to the distal extremity of the portion 231b by suitable means such as an adhesive. In order to increase the pushability of the portion 231c of the flexible elongate tubular member 231 while retaining its flexibility, a coil spring 236 is embedded within the plastic forming the portion 231c. The coil spring 236 is provided with a plurality of turns 237 as shown in detail in FIG. 15, which preferably are immediately adjacent or in apposition to each other to provide for maximum pushability. The coil spring 236 should extend at least throughout the length of the cylindrical member mounted coaxially thereover as hereinafter described. In addition, as shown the coil spring 236 can extend the entire length of the portion 231c. The coil spring 236 is carried by the portion 231c and preferably can be embedded or encapsulated within plastic 238 of the same type forming the tubular support member 231. Such embedding of the coil spring 236 prevents uncoiling of the coil elements or turns 237 and elongation of the flexible elongate tubular member 231 upon retraction of the inner elongate tubular member 231 into the outer elongate tubular member 226 with decrease in distance between proximal and distal ends of the expansion member. Alternatively, as shown in FIG. 16, a braided member 238 may be substituted for the coil spring 236 and also encapsulated or embedded with the plastic forming portion 231c. Such encapsulation also prevents elongation of portion 231 c upon retraction of the flexible elongate tubular support member 231 into the outer elongate tubular member 226. The metal braid 238 formed of a suitable material such as stainless steel wires 239 of a suitable diameter ranging from 0.0002″ to 0.003″ can be used to form the mesh for the braided member 238. The braided member 238 increases the pushability of the portion 231c of the inner flexible elongate tubular member 231 and also prevents substantial elongation of the inner flexible elongate tubular member 231. Furthermore, metal braid 238 can consist of flat ribbon.

A safety ribbon 241 is provided within the inner flexible tubular member 231 to prevent elongation of the portion 231c of the inner flexible elongate tubular member 231 and extends from the distal extremity of portion 231b to the distal extremity of portion 231c. The safety ribbon 241 can be formed of a suitable material such as stainless steel having a diameter area of 0.002″ to 0.004″ or a ribbon with a flat cross section. The safety ribbon 241 is disposed adjacent the portion 231c of the flexible elongate tubular member 231, and preferably as shown extends interiorly of the portion 231c in the lumen 234 and has its distal extremity secured to the distal extremity of the portion 231 c by solder 242. The safety ribbon 241 has its proximal extremity secured to the distal extremity of the portion 231 b of the inner flexible elongate tubular member 231 by the use of solder 242 (see FIG. 13).

An expansion member 246 is provided with proximal and distal extremities 247 and 248 as shown in FIG. 13 and is disposed coaxially on the portion 231 of the inner flexible elongate tubular member 231. The expansion member 246 is constructed in a manner similar to the expansion member 31 hereinbefore described and is provided with a plurality of flexible elongate elements or filaments 251 in which a plurality of elements 251 have a first common direction of rotation about the central axis as shown in FIG. 10 and are axially displaced relative to each other and cross over a further plurality of the flexible elongate elements 251 also axially displaced relative to each other but having a second common direction of rotation opposite to that of the first direction of rotation to form a double helix, braided or mesh-like cylindrical expansion member 246 with the crossing of the flexible elongate elements 251 occurring in the area of contact between the flexible elongate elements 251 to form openings or interstices 252 therebetween. The solder 242 used for securing the safety ribbon 238 to the coil spring 236 is also used for securing the distal extremity 248 of the cylindrical member 246 to the distal extremity of the inner flexible elongate tubular member 231. A sleeve 253 of heat shrink tubing covers the solder 242.

In order to increase the radial forces generated by the expansion member 246, it has been found that it is desirable to provide undulations 256 in which there is an undulation 256 present at each cross-over point of the filaments 251. Thus, as shown in FIG. 17, which is a fragmentary view of the cylindrical expansion member 246 shown in FIG. 13, an undulation 256 is provided in each of the plurality of flexible elongate elements 251 having a first direction of rotation at every other cross-over point with the plurality of flexible elongate elements having a second common direction of rotation about the central axis and wherein the undulations in the adjacent elements 251 are offset by one cross-over point so that in the resulting mesh or braid construction, the undulations 256 in one of the elements 251 having a first direction of rotation overlies every other cross-over point of the element 251 having a second direction of rotation and, conversely, every element 251 having a second direction of rotation has an undulation 256 therein at every other cross-over point of the elements 251 having a first direction of rotation. These undulations 256 can be in the form of obtuse angle bends having straight portions extending from both sides of the bend, or alternatively can be in the form of arcuate portions having a diameter corresponding generally to the diameter of the elements 251. Thus, it can be seen that the undulations 251 make it possible for one of the elements 251 to support the other of the elements at each cross-over point, thereby preventing slippage of the elements 251 with respect to each other and thereby causing greater radial forces to be applied when the cylindrical expansion member 246 is expanded as hereinafter described. Furthermore, alternate braid configurations can be employed. One such alternate configuration is two wires crossing two wires alternatively (known as a 2 over 2 braid),

The expansion member 246 is comprised of 16-64 individual elements 251 formed of 0.001 to 0.005 inch diameter wire of a suitable metal such as stainless steel helically wound around a longitudinal central axis. The helices are wound in opposite directions. Stretching or elongation of the cylindrical expansion member 246 results in a reduction in diameter of the expansion member 246. Mechanical fixation of the proximal and distal extremities 247 and 248 of the expansion member 246 holds these extremities in reduced diameter configurations. The positions of the elements 251 in these extremities cannot change in relation to each other. Therefore, the crossing angles of the elements 251 remain constant. Shortening of the cylindrical expansion member 246 with the ends fixed results in the formation of a cylindrical center section of great rigidity with the elements 251 in close apposition to each other. The tapered proximal and distal extremities of the expansion member 246 causes the stresses on the individual elements 251 to be balanced. Since the proximal and distal extremities 247 and 248 are held in constant tapered positions, the interstices 252 between the elements 251 are maintained allowing blood to flow into and out of the cylindrical center section when the expansion member 246 is shortened as shown in FIG. 18. Shortening of the expansion member or spring 246 results in a significant increase in the metal density per unit length in the center portion of the expansion member 246 while the metal density at the ends is relatively constant. This increase in metal density in the center section results in significant radial force generation as the elements 251 are compressed in a longitudinal direction into preformed diameters.

Use of the helically wound coil spring 236 or the braid 238 which serves with or as part of the inner elongate tubular member 231 and coaxially disposed within the cylindrical expansion member 246 provides greatly improved pushability and axial column strength for causing elongation of the cylindrical expansion member 246 while providing the desired flexibility so that tortuous curves can be negotiated during deployment of the mechanical dilatation and irradiation device 221. The portion 231c of the flexible elongate tubular member 231, and particularly within the cylindrical expansion member 246, has a relatively small diameter so that it does not adversely affect the stenosis crossing profile for the mechanical dilatation and irradiation device 221. The use of the inner or safety ribbon 241 prevents undue elongation and unwinding of the coil spring 236 forming a part of portion 231c of the flexible elongate tubular member 231 when the cylindrical expansion member 246 is lengthened or elongated. The pull or safety ribbon 241 also limits elongation of the cylindrical expansion member 246 and thereby prevents the elements 251 from being broken off or pulled away from the solder joints 253.

The proximal extremity 223 of the outer flexible elongate tubular member 222 of the mechanical dilatation and irradiation device 221 is provided with control means 261 for causing relative movement between the first or outer flexible elongate tubular member 222 and the second or inner flexible elongate tubular member 231 and can be similar to that hereinbefore described. This control means 261 consists of a fitting 262 which is bonded to the proximal extremity 223 of the outer flexible elongate tubular member 222. The fitting 262 is provided with a male Luer fitting 263 removably mated with a female Luer fitting 264 carried by a Y-adapter 266 which is provided with a central arm 267 and a side arm 268. The side arm 268 is in communication with the lumen 226 of the outer flexible elongate tubular member 222. The inner flexible elongate tubular member 231 extends through the central arm 267 of the y-adapter 266. A rotatable knob 269 is provided on the central arm of the y-adapter 266 for forming a fluid-tight seal between the central arm 267 and the portion 231 a of the inner flexible elongate tubular member 231. A male Luer fitting 271 is mounted on the proximal extremity of the portion 231a. The guide wire 26 extends through the lumen 234 of the inner flexible elongate tubular member 231 and extends beyond the distal extremity thereof.

As hereinbefore described, the control means 261 can include means such as a screw mechanism for causing relative movement between the outer flexible elongate tubular member 222 and the inner flexible elongate tubular member 231. Operation and use of the mechanical dilatation and irradiation device 221 is substantially similar to that hereinbefore described with respect to the previous embodiments. The mechanical dilatation and irradiation device 221 however has a number of features which may be more advantageous in certain medical procedures. Thus in medical procedures where improved pushability and torquability is required the use of the metal hypotube for the portion 231b of the flexible elongate tubular member provides additional pushability and torquability for the catheter facilitating advancement of the mechanical dilatation and irradiation device 221 through more difficult stenoses, particularly where additional torquability and pushability are desired. This is also true with the distal extremity of the mechanical dilatation and irradiation device 221 in which the inner flexible elongate tubular member 231 has the distal portion 231c thereof that includes the compressed coil spring 236 or braided member 238 which extends at least through the expansion member 246 to provide additional pushability for the expansion member 246 while still retaining the desired flexibility. Even though improved pushability is provided, the distal extremity of the mechanical dilatation and irradiation device 221 is still very flexible permitting it to track tortuosities in the vessels being negotiated thereby. Also because of the pushability of the inner flexible elongate tubular member 231, it is possible to obtain maximum extension of the expansion member 246 and thereby a minimum diameter to facilitate crossing of a stenosis with very small openings therethrough with the mechanical dilatation and irradiation device 221. The safety ribbon 241 prevents undue elongation of the inner flexible elongate tubular member 231. In addition, encapsulation of the compressed coil spring 236 or braided member 238 also prevents elongation of the inner flexible elongate tubular member 231.

When the expansion member 246 is being expanded by decreasing the length of the same, such as in the manner shown in FIG. 17, the diameter of the expansion member is increased to its maximum size with great rigidity because of the undulations 256 provided in the elements 251 of the expansion member 246. These undulations 256 aid providing greater radial forces while still retaining the conical or tapered ends with the open interstices to readily permit blood to pass through the expansion member 246 during the time that the expansion member 246 has been expanded to its maximum diameter to apply maximum radial forces to the stenoses which is being dilated during the procedure.

FIG. 19 depicts the mechanical dilatation and irradiation device with a series of bands 280 secured to the inner member which is either fabricated from a radioactive alloy or coated with a radioactive material. Any of the standard practices for activating a base material to a radioactive state known by those skilled in the art can be utilized or employed with the present invention.

The radioisotope used for these purposes (i.e. either incorporating the radioisotope into the material or subsequently activating the material by exposure to radiation source) may be an alpha, beta or gamma emitter or any combination of these. For clinical applications, a radioactive emitter with a half-life in the range between 10 hours and 50 days would be ideal. In addition, an ideal characteristic of the emitting radiation is that it does not travel long distances from the source, being absorbed by the tissue that is in close proximity to the radiation source. Furthermore, modest levels of radiation are desirable since significant levels of radiation are well known to damage the non-proliferative cells.

An example of an isotope that could be alloyed into the base material or subsequently activates the radioactive bands 280 is phosphorus 32, a beta emitter with a half-life of approximately 14 days. Another example for an ideal radiation emitter would be vanadium 48 another beta emitter with a half-life of approximately 16 days but which also emits a small portion of it total energy as gamma radiation. Other potential radioisotopes are platinum 125 which emits both beta particles and gamma rays with a half-life of 4.1 days. An example of a potential gamma ray emitter is iridium 189 with a half-life of 12 days.

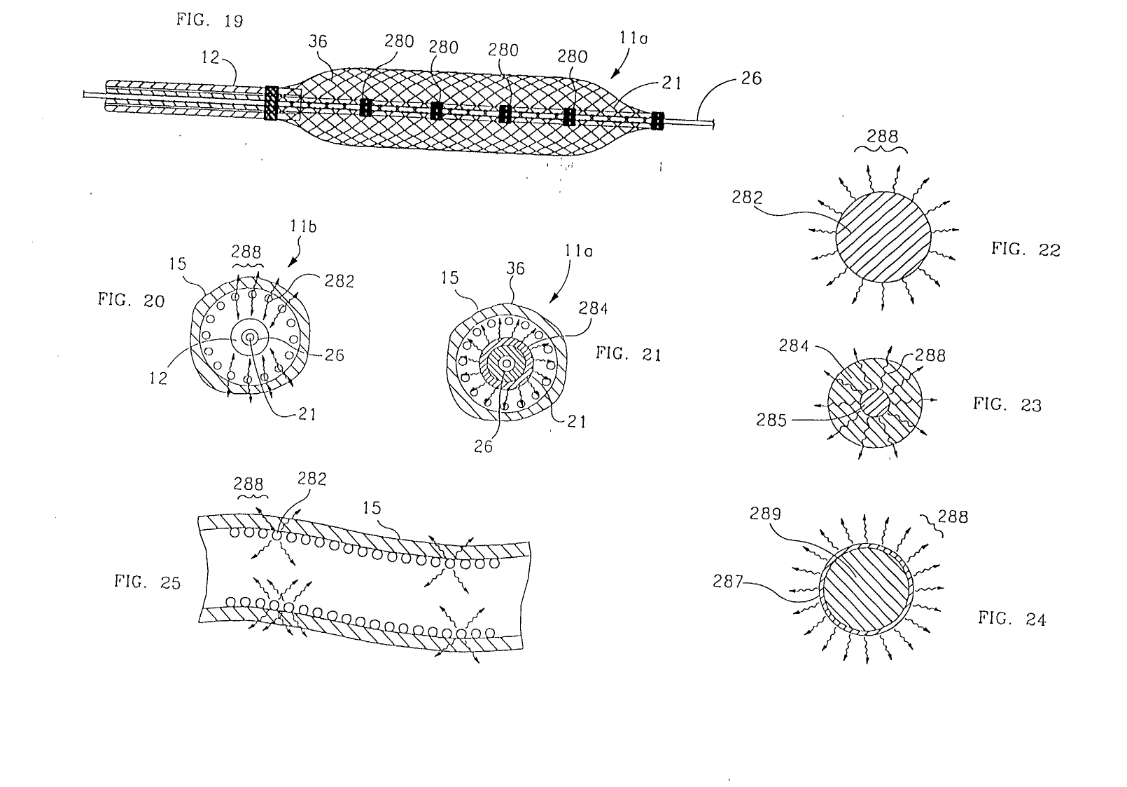

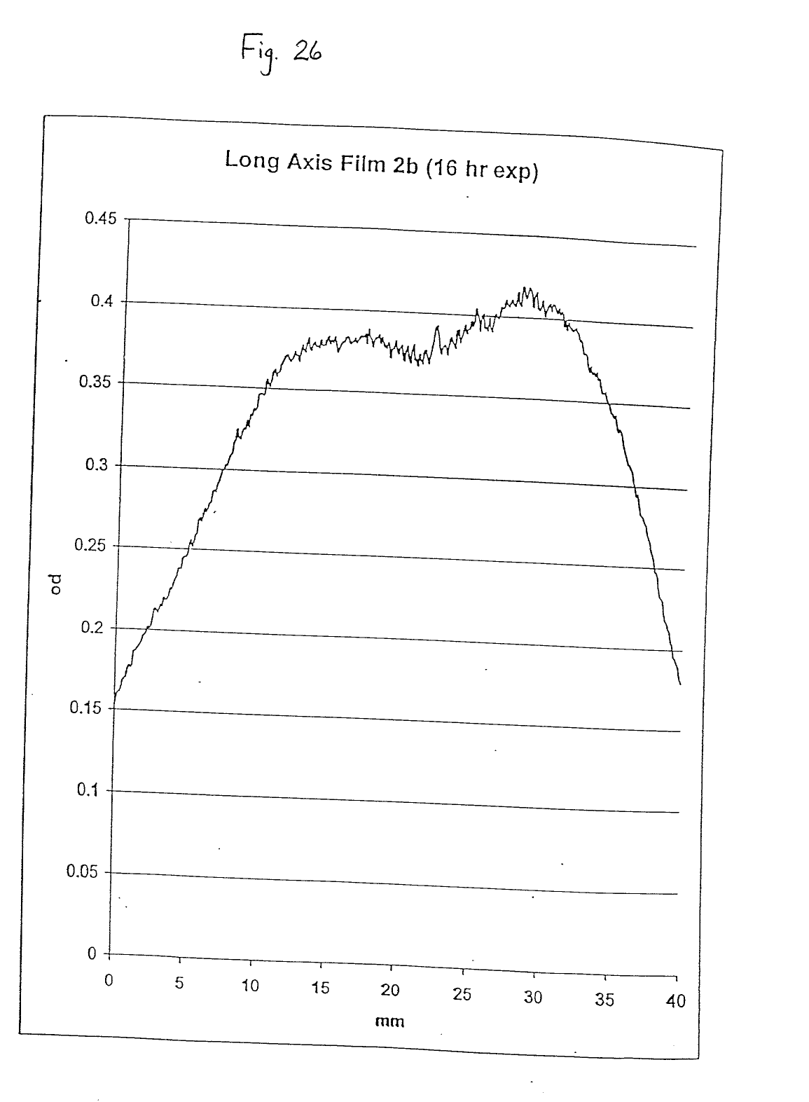

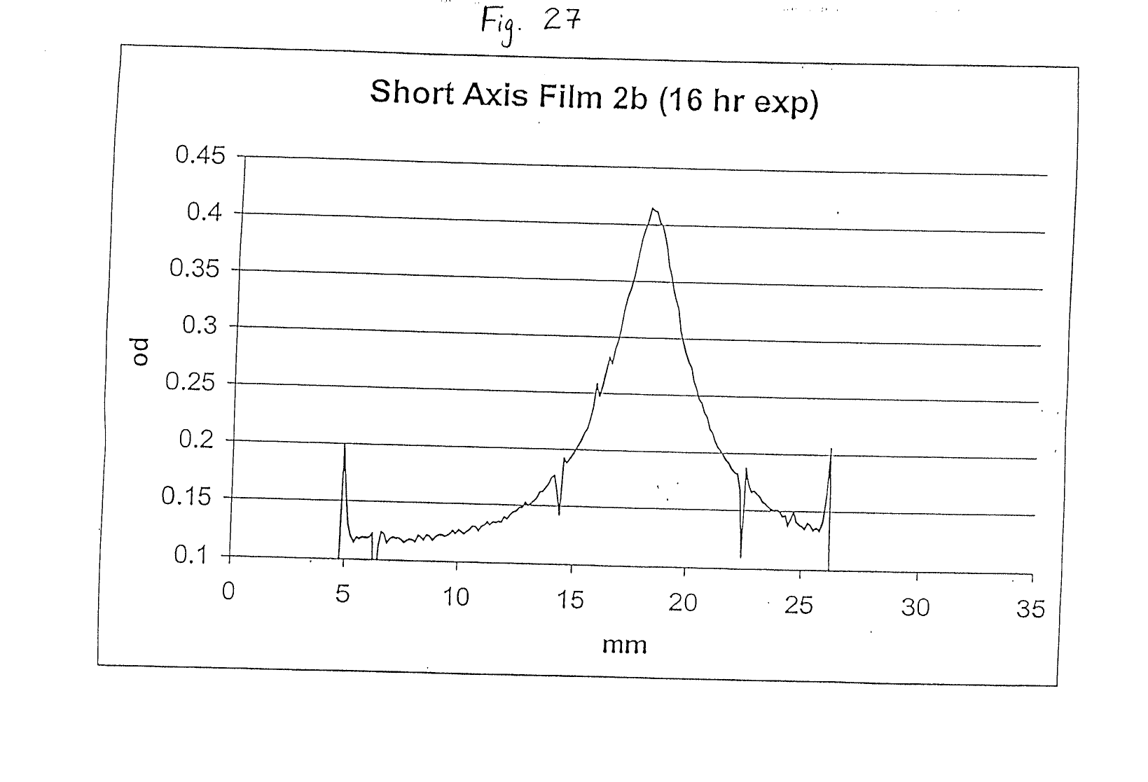

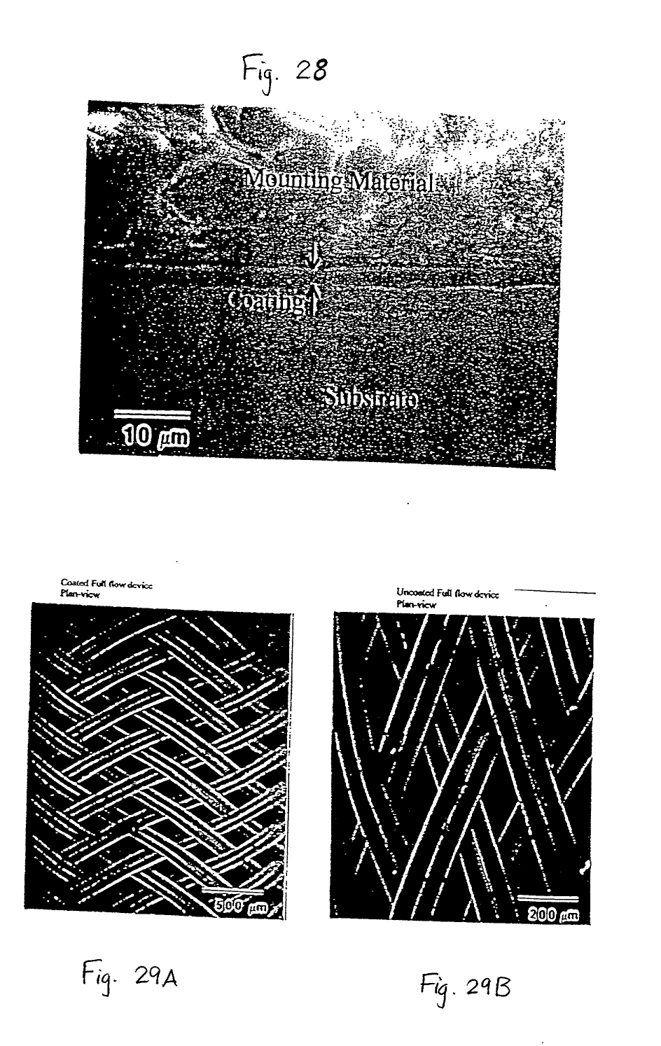

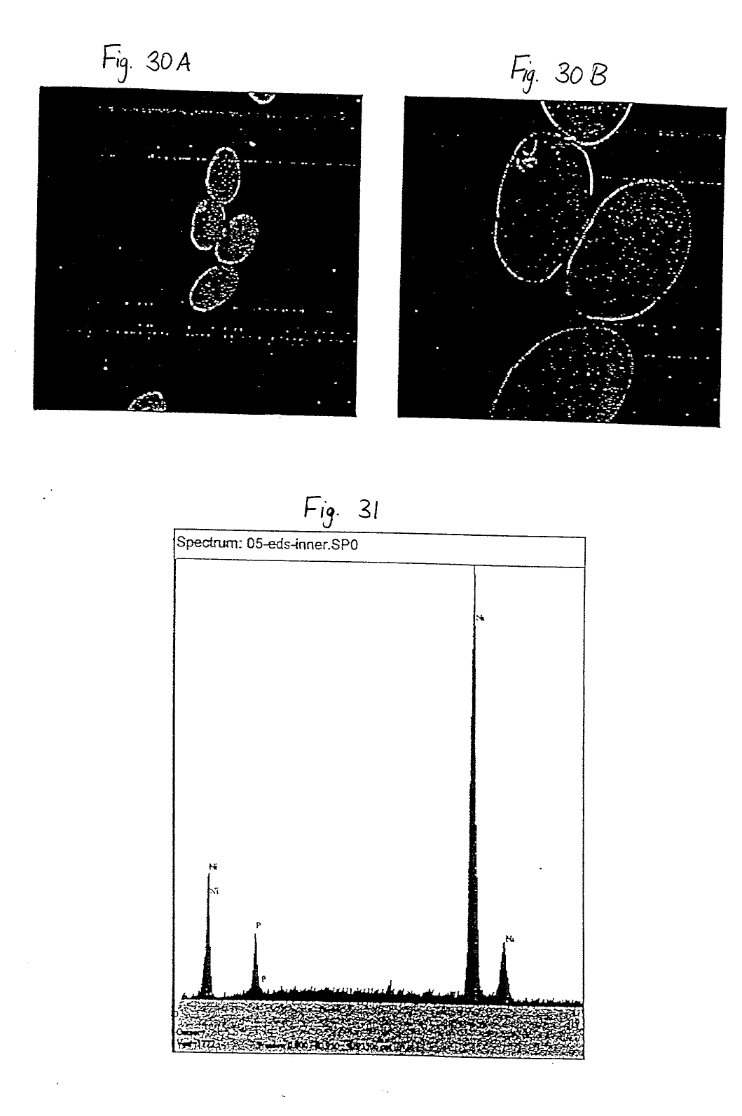

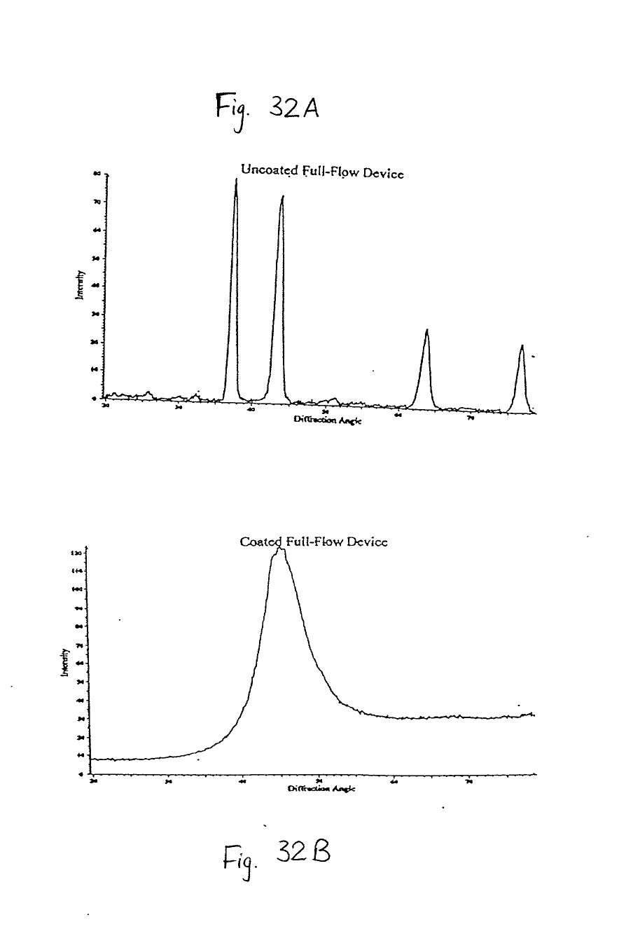

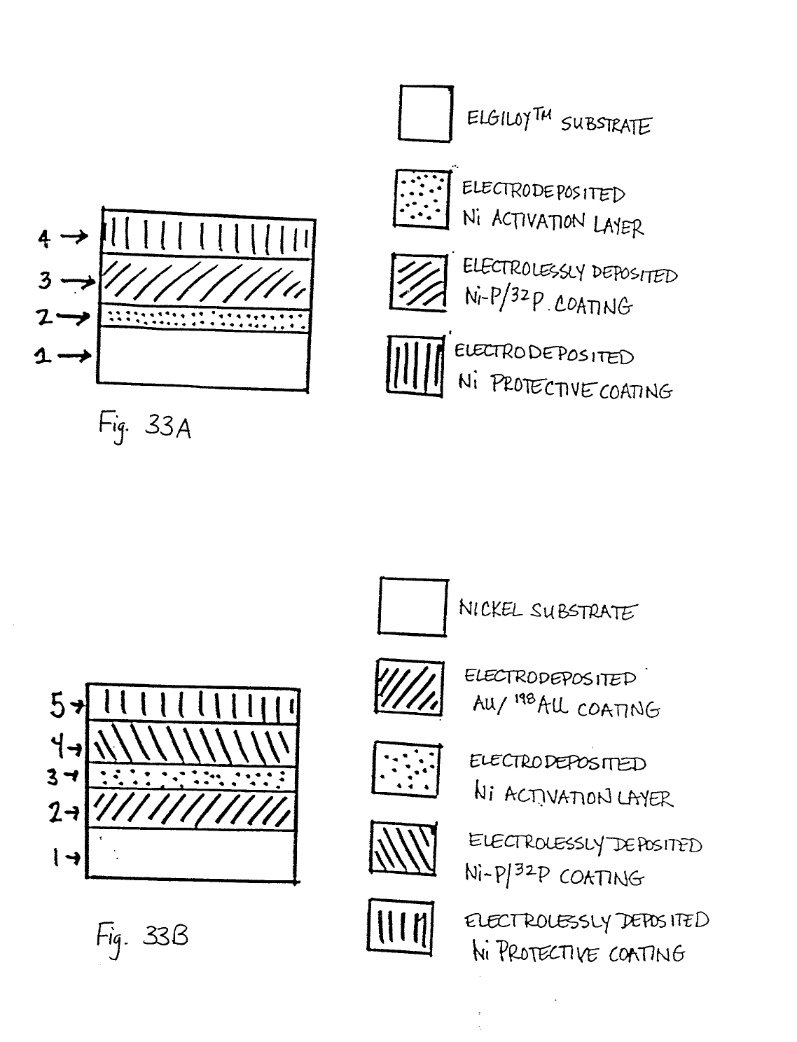

Alternatively, the entire inner member of the expandable mesh can be a radioactive source by utilizing either of the means described above. FIG. 21 is a cross-sectional view of FIG. 19 showing either a single radioactive band 284 or the entire inner tubular member as the radiation source. FIG. 21 also demonstrates the uniform path an alpha or beta particle, or gamma ray would project from these sources to the vessel wall 15.

In another embodiment of the present invention, the flexible elongate elements 282 of the present invention can themselves be the radioactive source as shown in FIG. 20. As demonstrated in the cross-sectional view of a flexible elongate element 282 (see FIG. 22), the element can be alloyed with or subsequently activate a non-radioactive material to emit radiation 288 uniformly to the vessel wall. As discussed above for the inner member or the bands which surround the member, the alloyed radioactive material can be represented by a number of isotopes. Also, as discussed above, any of the standard practices for activating a base non-radioactive material to a radioactive state known by those skilled in the art can be utilized or employed with the present invention.

Alternatively, as shown in FIG. 23, the flexible elongate element 284 can have a hollow core 285 that is filled with a solid, liquid or gaseous material that either is radioactive or with a non-radioactive solid, liquid or gaseous material that can subsequently become radioactive by standard activation mechanisms. The radioactive elongate element will emit radiation 288 uniformly to the vessel wall 15 as demonstrated in FIG. 23.

A further alternate is shown in FIG. 24 where the flexible elongate element is coated with a non-radioactive material that becomes radioactive by one of the well known activation mechanisms yielding a radioactive elongate element 289. Alternatively, a coating comprising a radioactive material 287 can be applied to the flexible elongate element rendering the present invention radioactive. The uniform distribution of radioactive alpha or beta particles or gamma rays 288 is demonstrated in cross-section in FIGS. 20 and 25.

An additional embodiment not shown but contemplated by the applicant is to utilize the present invention with a radioactive guidewire that can be inserted through the internal lumen 26 of the over-the-wire design or through the distal lumen 107 of the rapid exchange design to irradiate an obstruction while and subsequent to the dilatation procedure. The advantages of using the present invention include exposing a vascular segment or obstruction to an intravascular radiation source for prolonged periods while allowing continuous perfusion of blood into the distal to the treatment area. Furthermore the embodiment is capable of providing a uniform dose of radiation to the vascular segment by centering the radioactive guidewire in the vessel lumen.

From the foregoing, it can be seen that there has been provided a mechanical dilatation and irradiation device which can be used in the same manner as a balloon catheter in dilating a vessel segment or deploying a stent during an interventional procedure with the outstanding advantage that blood can continue to flow to the distal blood vessel during the procedure. This permits a longer vessel dilatation and irradiation without tissue ischemia. In addition, perfusion of side branches continues through the flexible cylindrical member. Furthermore, the dilatation and irradiation device provides delivery of a uniform dose to the affected vessel walls via either radiation delivered from the radioactive expansion member or from the radioactive flexible elongate elements centered within the vessel while the distal mesh is in its expanded state. Furthermore, the mechanical dilatation and irradiation device also provides the advantages of known expanded non-compliant diameter and therefore exact sizing. In addition, there is no possibility of a balloon rupture with leakage of radioactive materials due to protrusions from the surface of the stent or prosthesis perforating the balloon during deployment.

To carry out the invention described above, the inventors have discovered a method of rendering radioactive the dilatation/perfusion device previously described which utilizes a radioactive coating solution comprising at least one carrier metal ion and a radioisotope. In a particular embodiment, the radioactive coating solution comprises a carrier metal ion, a radioisotope and a reducing agent. Suitable carrier metals ions include, without limitation, nickel, copper, cobalt, palladium, platinum, chromium, gold and silver ions. In one embodiment of the present invention, the carrier metal ion is nickel ion. The concentration of carrier metal ion in the radioactive coating solution may vary, as would be understood by one skilled in the art. A representative carrier metal ion concentration is from about 1 to about 30 g/L. Carrier metal ion concentrations from about 3 to about 15 g/L are particularly suitable for use with radioactive coating solutions wherein the carrier metal ion is nickel.

Radioisotopes suitable for use in the coating solution of the present invention include beta, gamma, or alpha emitters. In a particular embodiment, the radioisotope is a non-metal (e.g., ³²P). Beta radiation penetrates only a limited distance through human tissue, and is therefore particularly desirable for localized radiation therapy. Beta emitters suitable for use in the present invention include, but are not limited to, ¹⁴C, ³⁵S, ⁴⁵Ca, ⁹⁰Sr, ⁸⁹Sr, ³²P, ³³P, ³H, ⁷⁷As, ¹¹¹Ag, ⁶⁷Cu, ¹⁶⁶Ho, ¹⁹⁹Au, ¹⁹⁸Au, ⁹⁰Y, ¹²¹Sn, ¹⁴⁸Pm, ¹⁴9Pm, ¹⁷⁶Lu, ¹⁷Lu, ¹⁰⁶Rh, ⁴⁷Sc, ¹⁰⁵Rh, ¹³¹I, ¹⁴⁹Sm, ¹⁵³Sm, ¹⁵⁶Sm, ¹⁸⁶Rc, ¹⁸⁸Rc, ¹⁰⁹Pd, ¹⁶⁵Dy, ¹⁴²Pr, ¹⁴³Pr, ¹⁴⁴Pr, ¹⁵9Gd, ¹⁵³Gd, ¹⁷⁵Yb, ¹⁶⁹Er, ⁵¹Cr, ¹⁴¹Ce, ¹⁴⁷Nd, ¹⁵²Eu, ¹⁵⁷Tb, ¹⁷⁰Tm, and ¹⁹⁴Ir. In a particular embodiment, the radioisotope is ³²P.

Gamma emitters suitable for use in the present invention include, but are not limited to, the group comprising ¹³⁷Cs, ⁶⁰Co and ¹⁹²Ir. Similarly, suitable alpha emitters include, but are not limited to, the group comprising ²²⁶Ra and ²²²Rn. Other radioisotopes suitable for use in the present invention include, but are not limited to, ¹²⁵I, ¹⁹²Ir and ¹⁰³Pd.

In a particular embodiment, the coating solution of the present invention is prepared by adding a water-soluble phosphorus compound to the coating solution, wherein at least a fraction of the P is ³²P. Put another way, ³²P is present in the coating solution as an aqueous solution of phosphorous-containing ions. In a particular embodiment, the source of ³²P is any compound containing hypophosphite (H₂PO₂). Non-limiting examples of hypophosphite compounds suitable for use in the present invention include hypophosphorus acid, sodium hypophosphite, ammonium hypophosphite, potassium hypophosphite and lithium hypophosphite. In a particular embodiment, aqueous NaH₂PO₂·H₂O or NaH₂PO₄·2H₂O is added to the coating solution, wherein at least a fraction of the P is in the form of ³²P.

In a further embodiment of the present invention, the source of ³²P is any compound containing phosphite (HPO₃²⁻). Phosphorous acid, H₃PO₃, provides a non-limiting example of a phosphite material suitable for use in the present invention. In a still further embodiment of the present invention, the source of ³²P is any compound containing orthophosphate (PO₄³⁻). Orthophosphoric acid, H₃PO₄, provides a non-limiting example of an orthophosphate compound suitable for use in the present invention.

The amount of radioisotope present in the radioactive coating solution may vary, as would be understood by one skilled in the art. A representative specific activity is from about 0.1 to about 5000 Ci/g, and more particularly, about 20 Ci/g (or 64/Ci/mole) which amount is particularly suitable for coating solutions comprising ³²P in the form of hypophosphite or hypophosphorus acid. This representative specific activity falls below the theoretical maximum for ³²P (i.e., slightly greater than 9000 Ci/mol, or 9,000,000 Ci/mol). This representative amount is particularly suitable where NaH₂PO₂·H₂O is the only reductant present in an electroless Ni—P coating solution.

Suitable reducing agents for use in the coating solution of the present invention include, but are not limited to, hypophosphites, formaldehyde, borohydride, dialkylamine boranes (e.g., dimethyl borane), and hydrazine. Each of these reductants has a particular condition range that is well known to one skilled in the art. In particular, for ENP, NaH₂PO₂ is commonly used as a reductant, with a representative range from about 5 g/l to about 50 g/l.

As would be evident to one skilled in the art, the radioisotope of the coating solution may be the radioactive form of an element present as the reducing agent, or a component thereof. For example, in a given coating solution, the radioisotope may be ³²P while the reducing agent might be NaH₂PO₂. Alternatively, the radioisotope may be the radioactive form of the carrier metal. For example, in a given coating solution, the radioisotope may be ¹⁹⁸Au, while the carrier metal is also Au.

In a particular embodiment of the present invention, the coating solution comprises NiSO₄ (26 g/l), NaH₂PO₂·H₂O (26 g/l), Na-acetate (34 g/l), lactic acid (18 g/l) and malic acid (21 g/l), wherein at least a fraction of the P is ³²P. In a further embodiment of the present invention, the coating solution comprises AuCN (2 g/L), NaH₂PO₂ (10 g/L), KCN (0.2 g/L), wherein at least a fraction of the P is ³²P. In a still further embodiment, the coating solution comprises AuCN (2 g/L), NaH₂PO₂ (10 g/L), KCN (0.2 g/L) wherein at least a fraction of the Au is ¹⁹⁸Au. In a still further embodiment, the coating solution comprises AuKCN (5.8 g/L), KCN (14 g/L), KOH (11.2 g/L), and KBH₄ (21.6 g/L), wherein at least a fraction of the Au is ¹⁹⁸Au.

Additional components may be added to the coating solution to vary the physical and chemical characteristics of the coating.

The present invention further relates to a method of forming a radioactive coating on a substrate, which coating comprises at least one carrier metal and a radioisotope. The coating is formed by contacting the substrate with a radioactive coating solution comprising a carrier metal ion and a radioisotope. The coating solution may have the properties described above, as one skilled in the art would appreciate. Various coating techniques known in the art are suitable for use in the present invention including, but not limited to, electroless deposition, electrodeposition, chemical vapor deposition, physical deposition, thermal spraying, sol-gel methods, or any combination thereof. Certain methods may be more suitable for certain substrates, as would be understood by one skilled in the art.

Substrates coated according to the present invention may include, but are not limited to, metals, alloys, polymers, plastics, ceramics and composites. As previously described, the substrate is medical device, such as a catheter, guidewire, stent or brachtherapy device (i.e., a hollow or solid needle), or a component thereof. In a more particular embodiment, the substrate is an expandable component of a catheter. This expandable component may be formed from a metal, alloy, polymer, plastic, ceramic or composite. In a particular embodiment, the expandable component is formed from an alloy, such as Elgiloy™.

In a particular embodiment of the method, electroless deposition is used to form a radioactive coating on a substrate. In this embodiment, the substrate is contacted with the radioactive coating solution, for a time, at a concentration, a temperature and pH sufficient to chemically deposit a radioactive metal coating on the substrate. It may be necessary to clean the substrate and to remove surface oxides therefrom prior to deposition of the radioactive coating. It may further be necessary to coat the substrate with a catalytic coating or activating layer prior to electroless deposition of the radioactive coating, as would be recognized by one skilled in the art. The catalytic coating may be a non-radioactive Ni coating, for example. Suitable electroless coating solutions include, without limitation, electroless nickel coating solutions comprising hypophosphite, wherein at least a fraction of the P in hypophosphite is ³²P. Typical electroless nickel coating solutions are reviewed in W.

Ying and R. Bank, Metal Finishing (December 1987), pp. 23-31, and in W. Riedel, Electroless Nickel Plating, ASM International (1991), pp. 9-32, which are incorporated herein by reference. Suitable electroless coating solutions also include electroless gold coating solutions comprising hypophosphite, wherein at least a fraction of the P in the hypophosphite is ³²P, as well as electroless gold solutions wherein at least a fraction of the Au is present as ¹⁹⁸Au. In a particular embodiment of the method, the radioisotope is the radioactive form of an element present as the reducing agent, or a component thereof (e.g., the radioisotope is ³²P, and the reducing agent is Na₂H₂PO₂). In a further embodiment of the method, the radioisotope is the radioactive form of the carrier metal (e.g., the radioisotope is ¹⁹⁸Au, while the carrier metal is Au.)

Conditions for electroless deposition of a particular coating solution can vary, as would be recognized by one skilled in the art. These conditions also vary depending on the desired coating composition. Representative condition ranges include: (1) a pH range of from about 4.5 to about 10.0, and more particularly 4.8; (2) a temperature range of from about 60 to about 100° C., and more particularly 88° C.; (3) a metal concentration range from about 3 to about 15 g/L; (4) a deposition rate range of from about 0.5 to about 257 mil/hour, and more particularly 10 mil/hour; (5) a bath loading range of from about 0.1 to about 1.0 square feet per gallon, and more particularly 0.6 square feet per gallon; and (6) one or more reductants, from about 5 to about 50 g/L. A representative deposition of 1 μM at 10 μM/hour would take 6 minutes. These representative ranges are particularly suitable for use in electroless deposition of an electroless nickel-phosphorus coating solution having Na₂PO₂—H₂O as a reductant, wherein at least a portion of P is ³²P. Other suitable conditions for electroless nickel-phosphorus deposition are reviewed in Hur et al, Microstructures and crystallization of electroless Ni—P deposits, Journal of Materials Science, Vol 25, (1990), 2573-2584, which is incorporated herein by reference. Accurate temperature and metal concentration control are important to achieve uniform deposition rates. Various coating thicknesses are achievable, as would be apparent to one skilled in the art. A representative coating thickness ranges from about 0.1 to about 20 μM, and typically about 1.0 to about 2.0 μm. Optionally, a sealing or protective layer may be formed, i.e., a non-radioactive Ni sealing layer).

FIG. 33A depicts a substrate having an electroless radioactive coating. More particularly, this Figure depicts an Elgiloy substrate (1) coated by electrodeposition of a Ni activation layer (2), which activated substrate has a radioactive Ni—P/³²P layer (3) formed thereon. The radioactive coated substrate in FIG. 33 also has a Ni sealing layer electrodeposited thereon (4).

The method of the present invention also includes the use of electrodeposition to apply a radioactive coating on a substrate. According to this method, the substrate is contacted with a radioactive coating solution for a time, at a concentration, at a temperature and voltage sufficient to electrically deposit a radioactive metal coating on the substrate. In some cases, it may be necessary to clean the substrate surface and to remove surface oxides prior to coating. In a particular embodiment of the method, the radioisotope present in the coating solution is a non-metal (e.g., ³²P). In a more particular embodiment, the coating solution comprises hypophosphite, phosphite, and/or orthophosphate, wherein at least a fraction of the P in the hypophosphite and/or the phosphite, and/or the orthophosphate is ³²P.

Suitable coating solutions for use an electrodeposition of radioactive metal coatings include, without limitation, a solution comprising nickel sulfate (150 g/L), nickel chloride (45 g/L), sodium hypophosphite (100 g/L), and orthophosphoric acid (50 g/L), wherein at least a portion of the P present is ³²P. Though conditions for electrodeposition vary, as would be familiar to those skilled in the art, representative conditions for electrodeposition of this radioactive coating solution include (1) a pH of about 6.0 to about 7.0; (2) a temperature of about 55-60° C.; (c) a coating density from about 20 mA/cm² to about 500 mA/cm², and more particularly, about 80 mA/cm². In one embodiment, the method yields a dense, amorphous Ni—P coating, at a coating rate of 4.2 μm/h. Generally, for electrodeposited coatings, coating rates may vary considerably from, for example, about 0.1 to about 25 μm/hour using conventional electrodeposition. Various coating thicknesses are achievable, as would be apparent to one skilled in the art. A representative coating thickness ranges from about 0.1 to about 20 μM, and typically about 1.0 to about 2.0 μm. Optionally, a protective or sealing layer is formed onto the radioactive coated substrate, such as a non-radioactive Ni coating.

In a further embodiment, a radioactive coating solution suitable for electrodeposition comprises CrK(SO₄)₂·12H₂O (100 g/L), NiSO₄·6H₂O (50 g/L), (NH₄)₂SO₄ (50 g/L), NaH₂PO₂·H₂O (50 g/L), Na₃C₆H₅O₇·2H₂O (50 g/L),C₆H₈O₇ (25 g/L), H₃BO₃ (20 g/L), (NH₂)₂CS (0.01 g/L), C₁₀H₁₆O (0.333 g/L), and C₁₂H₂₅SO₄Na (0.1 g/L), wherein at least a fraction of the P in NaH₂PO₂·H₂O is present as ³²P. Representative conditions for electrodeposition of this solution include (1) pH from about 2 to about 4, and typically 2.3; (2) current density from about 5 to about 400 m A/cm2, and typically about 200 mA/cm2; (3) a temperature at or about room temperature.

In yet a further embodiment, a radioactive coating solution suitable for electrodeposition comprises NiSO₄ 6 H₂O, NiCl₂ 6 H₂O, NiCO₃, H₃PO₃, H₃PO₄ and saccharin, wherein at least a fraction of the P in H₃PO₃ is present as ³²P. Representative conditions for electrodeposition of this solution include (1) pH from about 0.75 to 0.90 and typically 0.82; (2) a current density from about 5 to about 400 mA/cm2, and typically about 20 mA/cm2; (3) a temperature between 40° C. and 95° C., typically 60° C.

The method of the present invention also includes the use of an applicator to apply a radioactive coating solution to the substrate. Suitable applicators include, but are not limited to, brushes and pens. Applicators for use in electroplating have an electrically conductive component. See U.S. Pat. No. 5,401,369 and U.S. Pat. No. 4,159,934.

In a particular embodiment of the present invention, the radioactive coating solution comprises at least one carrier metal ion and either an insoluble radioisotope or the insoluble compound of a radioisotope. In a particular embodiment, the radioactive solution also includes a reducing agent, with suitable reducing agents including identified above for radioactive coating solutions comprising at least one dissolved carrier metal ion and a dissolved radioisotope. The carrier metal ion is dissolved in solution, and may be, without limitation, nickel, copper, chromium, cobalt, platinum, palladium, gold or silver ion. In a particular embodiment, the carrier metal ion is copper, which can be dissolved in the coating solution in the form of any soluble copper salt, such as CuSO₄. In a further embodiment, the dissolved carrier metal ion is nickel.

The concentration of carrier metal ion in the radioactive coating solution may vary, as would be understood by one skilled in the art. A representative carrier metal ion concentration range would be from about 1 to about 30 g/L. Carrier metal concentrations from about 3 to about 15 g/L are particularly suitable for use with radioactive coating solutions wherein the carrier metal is nickel.

The insoluble radioisotope may comprise an insoluble radioisotope or insoluble compound of a radioisotope, such as an insoluble metal salt or oxide. Insoluble radioisotopes suitable for use in the coating solution of the present invention include, without limitation, insoluble ³²P, ⁹⁰Y and ¹⁹⁸Au. Insoluble compounds of radioisotopes include, without limitation, FeP, NiP, CoP, SnP, Ti₄P₃ and Y₂O₃, wherein ³²P, ¹²¹Sn or ⁹⁰Y are present in substantial amounts. Alternatively, the soluble compound of a radioisotope can be rendered insoluble, e.g., by encapsulation or immobilization in an insoluble coating or matrix. Various other metal oxides and metal phosphides are also suitable for use in the present invention.

The insoluble radioisotopes or insoluble compounds of radioisotopes may be in the form of metal or alloy particles, metal oxide particles, or polymeric particles. The size of the particles present in the coating solution may vary, as would be apparent to one skilled in the art. A representative particle size ranges from about 5 nm to about 30 μm. As a non-limiting example, ¹⁹⁸Au particles formed by wet grinding gold range from about 1 to about 10 μm in diameter are suitable for use in the present invention. In a particular embodiment of the present invention, the radioactive coating solution comprises particles of varying sizes. See U.S. Pat. No. 4,547,407.

The amount of insoluble radioisotope present in the radioactive coating solution may vary, as would be understood by one skilled in the art. A representative amount has a specific activity of about 0.1 to about 5000 Ci/g.

In a particular embodiment of the present invention, the coating solution comprises 1.0 mol/L CuSO₄, 0.75 mol/L H₂SO₄, and 35 mg/L P in particulate form, suspended in solution via agitation, wherein at least a fraction of the P is ³²P. In a further embodiment of the present invention, the coating solution comprises NiSO₄ (26 g/L), NaH₂PO₂—H₂O (26 g/L), Na-acetate (34 g/L), lactic acid (18 g/L), malic acid (21 g/L), and Au in particulate form, wherein at least a portion of the Au is ¹⁹⁸Au. Additional components may be added to the coating solution to vary the physical and chemical characteristics of the coating solution.

The present invention also relates to a method of forming a radioactive coating on a substrate, which coating comprises a metal matrix and a dispersed radioactive phase. The composite coating is formed by contacting the substrate with a radioactive coating solution comprising at least one carrier metal and either an insoluble radioisotope or an insoluble compound of a radioisotope. The radioactive coating solution may have the properties described above, as one skilled in the art would appreciate. Suitable coating techniques include, but are not limited to, electroless deposition, electrodeposition, chemical vapor deposition, physical deposition, thermal spraying, or any combination thereof. Certain methods may be more suitable for certain substrates, as would be understood by one skilled in the art.

The quantity of radioactive particles deposited onto the substrate is influenced by various factors, including (1) the concentration of radioactive particles in the coating solution; (2) particle size and distribution; and (3) coating conditions. It is generally necessary to agitate the coating solution during the coating process. Substrates coated may include, but are not limited to, metals, alloys, polymers, ceramics and composites. In a particular embodiment, the substrate may be a medical device, or a component thereof, formed of metal, alloys, polymers, ceramics or composites, or combination thereof. Representative medical devices, without limitations, include catheters, guidewires, stents and brachytherapy devices. In a particular embodiment, the substrate is an expandable component of a catheter. In a particular embodiment, the expandable component is formed from an alloy, such as Elgiloy™.

In one embodiment of the method of the present invention, the radioactive composite coating is formed on the substrate by electrodeposition. The use of electrodeposition to form composite coatings is discussed in U.S. Pat. No. 5,266,181. More particularly, the substrate to be coated is contacted with the coating solution of the present invention for a time, at a concentration, a temperature, a cathode current density, and inter-electrode distance sufficient to electrically deposit a radioactive Composite coating thereon. In some cases, it may be necessary to clean the substrate and to remove surface oxides therefrom prior to deposition of the radioactive coating. In a particular embodiment of the method of the present invention, the radioactive coating solution comprises 1.0 mol/L CuSO₄, 0.75 mol/L H₂SO₄, and a steady state concentration of 35 mg/L P in particulate form, suspended in solution via agitation, wherein at least a fraction of P is ³²P.

Electrodeposition conditions may vary from one coating system to another, as would be recognized by one skilled in the art. In a particular embodiment, electrodeposition of the Cu—P coating solution above is performed at a cathode current density of 18 mA/cm², an inter-electrode distance of 5 cm, at a temperature at or near 40° C. See J. W. Graydon and D. W. Kirk, “Suspension Codeposition in Electrowinning Cells: The Role of Hydrodynamics,” the Canadian Journal of Chemical Engineering, vol, 69 (1991) 564-570. Agitation of the insoluble radioisotope particles is necessary via stirring or alternatively via recycle flows (500-1000 mL/min) to achieve uniform deposition rates. Coating rates vary with current density, temperature and other bath parameters. Suitable coating thickness' range from about 0.1 to about 20 μm, with about 5 μm generally suitable.

In a further embodiment of the present invention, the radioactive composite coating is formed by electroless deposition. Electroless deposition of composite coatings is reviewed in U.S. Pat. Nos. 5,232,744 and 5,389,229. More particularly, the substrate to be coated is contacted with the coating solution comprising at least one carrier metal ion, an insoluble radioisotope or insoluble compound of a radioisotope, and a reducing agent, for a time, at a concentration, at a temperature and pH sufficient to chemically deposit a radioactive composite coating thereon. Electroless deposition conditions may vary, as would be apparent to one skilled in the art. A representative electroless deposition involves contacting the substrate with a coating solution comprising from about 0.5 to about 0.5 mol/l of a metal, from about 0.1 to about 0.5 mol/l of a reducing agent and about 0.1 to about 500 g/l of particulate matter, at least a fraction of which comprises a radioactive isotope, wherein the coating solution has a pH ranging from about 4 to about 8, at a temperature of about 50 to about 95° C., and more particularly 70-90° C., for a time dependent on the particular coating thickness desired. In this embodiment, the radioisotope present in the coating solution acts to reduce the metal present therein to deposit a metal layer on the substrate surface. Thickness of the coating may vary, and range from about 0.1 to about 20 μm, and typically from about 1 to about 2 μm. Optionally, the substrate to be includes a catalytic coating layer or activating layer is coated onto the substrate prior to coating with the radioactive coating. The catalytic coating layer may be an electrolessly deposited or electrodeposited Ni coating layer, for example.

In one embodiment of the method of the present invention, the radioactive coating solution comprises NiSO₄ (26 g/L), NaH₂PO₂·H₂O (26 g/L), Na-acetate (34 g/L), lactic acid (18 g/L), malic acid (21 g/L), and Au in particulate form, wherein at least a portion of Au is present as ¹⁹⁸Au. In a further embodiment, the radioactive coating solution comprises NiSO₄ (26 g/L), NaH₂PO₂·H₂O (26 g/L), Na-acetate (34 g/L), lactic acid (18 g/L), malic acid (21 g/L), and Y₂O₃ in particulate form, wherein at least a fraction of the Y in Y₂O₃ is ⁹⁰Y.

In a still further another embodiment, the coating solution comprises NiSO₄ (26 g/L), NaH₂PO₂·H₂O (26 g/L), Na-acetate (34 g/L), lactic acid (18 g/L), malic acid (21 g/L), and a polymer phosphate in particulate form, wherein at least a fraction of the P in is ³²P. Polymers containing phosphorus are reviewed in Nakano et al. (JP# 11061032). For example, Nakano describes preparation of a 2-hydroxyethyl methacrylateltert-Bu methacrylate/ethyl methacrylate phosphorylated with phosphorus oxychloride or polyphosphoric acid to form a polymer phosphate. In one embodiment of the present invention, a portion of the P in the phosphorus oxychloride or polyphosphonic acid is ³²P, and the resulting radioactive polymer phosphate is powder processed to form a mean particle size ranging, for example, from about 5 to about 30 μM. The radioactive polymer particles are then incorporated into the coating solution. All nonsoluble particles are kept in solution by means of intensive mechanical mixing (e.g., 300 rpm).

One advantage of the composite coating solution of the present invention is the ability to separate the radioactive source from the coating solution, e.g., by filtration. Separation makes it unnecessary to treat and dispose of the entire volume of the coating solution as radioactive waste, limiting the expense of waste treatment. According to this embodiment of the coating solution of the present invention, recharging, of isotopes is permissible, providing an economic advantage.

The present invention also relates to radioactive sols and sol-gels, and to radioactive coatings formed via sol-gel techniques. The radioactive sol-gel of the present invention comprises an oxide and a radioisotope. Sol-gel techniques are reviewed generally in Pierre, A., Introduction to Sol-Gel Processing (1998), which is incorporated herein by reference. The radioactive sol-gel of the present invention may be formed via either colloidal or polymeric routes, resulting in either a polymeric or a colloidal radioactive sol-gel. A discussion of polymeric and colloidal gels and synthesis routes is found in C. D. E. Lakeman and D. A. Payne, Invited Review: Sol-gel Processing of Electrical and Magnetic Ceramics, Materials Chemistry and Physics, 38 (1994) 305-324, which is incorporated herein by reference.

Formation of both the colloidal and polymeric radioactive sol-gels of the present invention involves the dissolution of a metal ion, either as alkoxides or as another organometallic compound in a suitable solvent to form a fluid sol. The metal alkoxide or other organometallic compound hydrolyzes, either partially or completely, and then polymerizes, resulting in gelation and the formation of a radioactive semi-rigid gel, known as a sol gel. The radioisotope present in the sol may be either soluble or particulate (insoluble). The specific activity of this radioisotope ranges, for example, from about 0.1 to about 5000 Ci/g. Metal alkoxides suitable for use in the present invention include, but are not limited to, alkoxides of metals including silicon, boron, zirconium, titanium and aluminum. In particular, the metal alkoxide is silicon alkoxide.

In one embodiment of the present invention, a polymeric radioactive sol-gel is formed from a sol comprising a metal alkoxide and a radioisotope, which metal alkoxide hydrolyzes and then polymerizes to form a radioactive sol-gel. In a particular embodiment of the present invention, the radioactive sol-gel is formed by reacting orthophosphoric acid with silicon alkoxide, wherein at least a fraction of the P is ³²P, to form a soluble, substantially linear polymer having P—O—Si linkages. This polymer is converted to a cross-linked polymer in the presence of sufficient water.

The radioactive sol-gel may also be formed via a colloidal route. Thus, in a particular embodiment, a Fe—P—O sol-gel may be formed according to the method described by Yamaguchi et al, in IEEE Transactions on Magnetics, 25 (1989) 3321-3323, incorporated herein by reference, wherein at least a portion of the P is ³²P in the present invention.

In a particular embodiment, the radioisotope present in the sol-gel comprises an insoluble radioisotope or compound of a radioisotope. The formation of sol-gels comprising insoluble components is reviewed in Nazeri et al., Ceramic Composites by the Sol-Gel Method: A Review, Chemical Engin. Sci. Proc. 14[11-12] (1993), pp. 1-19, the contents of which are incorporated by reference. In a particular embodiment of the method, the sol comprises a metal alkoxide and an insoluble radioisotope, which metal alkoxide hydrolyzes, either partially or completely, and then polymerizes to from a radioactive sol-gel having insoluble radioisotope dispersed therein. In a further embodiment of the present invention, the sol comprises a metal alkoxide, which hydrolyzes and polymerizes to a state short of gelation, providing a partially polymerized sol which is then impregnated with the insoluble radioisotope. The impregnated sol then further polymerizes to produce a radioactive sol-gel having an insoluble radioisotope dispersed therein. In another embodiment of the present invention, a sol comprising a metal alkoxide is hydrolyzed and polymerized to form a sol-gel, which is then impregnated with the insoluble radioisotope to produce a radioactive sol-gel having insoluble radioisotope dispersed therein.

In a particular embodiment of the present invention, a sol is prepared by hydrolysis of tetra orthosilicate (TEOS) with radioactive particles (e.g., Au/¹⁹⁸Au) or (P/³²P) mixed therein. The concentration of these particles may vary as would be recognized by those skilled in the art, with a representative activity from about 0.1 to about 5000 Ci/g.

The present invention also relates to methods of forming radioactive coatings onto substrates by sol-gel techniques. In a particular embodiment of the method, the substrate to be coated is contacted with a radioactive sol comprising a metal alkoxide or an organometallic compound and a radioisotope. The sol hydrolyzes and polymerizes to produce a radioactive sol gel on the substrate. This radioactive sol-gel is then dried, and optionally subjected to high temperature treatments that (a) may remove volatile species, including but not limited to hydroxyl groups or residual organic groups; and/or (b) result in processes which produce shrinkage and removal of residual porosity, including but not limited to sintering; and/or (3) result in processes that involve phase changes, including but not limited to crystallization and chemical reactions. The dried, and optionally high temperature treated, sol-gel forms a radioactive oxide coating comprising an oxide and a radioisotope. In a particular embodiment, the sol has undergone polymerization to a certain state, short of gelation, prior to being coated onto the substrate. Put another way, a partially polymerized sol is coated onto the substrate.

In a particular embodiment of the present invention, the substrate is contacted with a radioactive sol formed by reacting orthophosphoric acid with silicon alkoxide, wherein at least a fraction of the P in the orthophosphoric acid is ³²P, as described above. Following hydrolysis and polymerization, a radioactive sol-gel is present on the article. The sol-gel is dried and optionally densified and crystallized to form a phosphorus silicon oxide coating containing ³²P.

In another embodiment, a radioactive coating is formed by spin-coating a substrate with the radioactive Fe—P—O sol described above, where the sol has an appropriate viscosity (i.e., about 80 co). The radioactive coating is then dried in air at 200° C. Following drying, an optional heat treatment may be conducted to crystallize the gel into a polycrystalline ceramic coating. For example, heating for 24 hours at 600° C. crystallizes the coating.

The present invention also relates to a method of forming radioactive composite coating by sol-gel processes. In a particular embodiment of the method, a substrate is contacted with a radioactive sol comprising a metal alkoxide or another organometallic compound and an insoluble radioisotope. In a particular embodiment, the radioactive sol comprises hydrolyzed tetraethyl orthosilicate (TEOS) with ³²P in particulate form dispersed therein, as described above. After the substrate is coated (i.e., by dipping or spin coating) with the sol containing a radioactive dispersed phase, it is dried to form a radioactive composite coating comprising an oxide matrix and a radioactive dispersed phase. The sol used to coat may or may not have undergone polymerization to a state short of gelation. Optionally, the radioactive coating is densified and crystallized into a crystalline ceramic article. During crystallization, the dispersed phase may optionally react/combine with the silica matrix, and consequently, the radioactive material may not appear to exist as a separate dispersed phase in the crystallized ceramic coating.

In a further embodiment of the method, a sol is formed comprising a metal alkoxide or another organometallic compound, and undergoes polymerization to a state short of gelation. An insoluble radioisotope is then introduced either into the partially polymerized sol, forming a radioactive partially polymerized sol which is then coated onto a substrate. In another embodiment of the present invention, a sol is formed comprising a metal alkoxide or another organometallic compound, and coated onto a substrate to form a sol-gel. This sol-gel is then impregnated with an insoluble radioisotope. Surface coating or full impregnation of the sol-gel can be achieved using this technique. The radioactive sol-gel is then dried and optionally crystallized into a crystalline ceramic structure.

The present invention is also directed to a method of forming multiple layers of a radioactive coating or coatings onto a substrate. Coating techniques suitable for forming such layers include, without limitation, electroless deposition, electrodeposition and sol-gel methods. According to one embodiment of the method, the substrate is contacted with a first radioactive coating solution under conditions sufficient to deposit a radioactive coating thereon. Optionally, the substrate is coated with a catalytic coating layer prior to deposition of the radioactive coating layer (i.e., a Ni activation coating layer). The substrate comprising a first radioactive coating is then contacted with one or more additional radioactive coatings solutions under conditions sufficient to deposit one or more additional radioactive coating layers thereon, thereby forming a substrate two or more radioactive coating layers. This process can be repeated to provide a substrate having multiple layers of radioactive coatings. Optionally, the coated substrate is rinsed prior to being contacted with the one or more additional radioactive coating solution, and/or between deposition of these additional radioactive coatings. Optionally, one or more catalytic/activation coating layers or activating layers may be coated onto the substrate and/or between one or more of the additional radioactive coating layers.

According to another embodiment of the present invention, the substrate is coated with a radioactive sol under conditions sufficient to deposit a radioactive sol-gel coating thereon. In a particular embodiment, the radioactive sol may be at least partially polymerized. The coated substrate is then coated with one or more additional radioactive sols under conditions sufficient to deposit a one or more additional radioactive sol-gel coatings thereon. This process can be repeated to provide a substrate having multiple layers of radioactive coatings.

The multiple radioactive coating layers of the present invention may be the same or different. For example, radioactive coating layers comprising soluble radioisotopes may be present or alternate with composite radioactive coating layers having a radioactive dispersed phase, while radioactive coating layers formed by electrodeposition may be present or alternate with radioactive coating layers formed by electroless deposition or sol-gel processes, and variations thereof. The radioisotope and/or the carrier metal present in alternating radioactive coating layers may be the same or different. In one embodiment of the method, the first radioactive coating layer is different than one or more additional radioactive coatings layers. For example, the radioisotope of the first radioactive coating layer may be different than the radioisotope of one or more of the additional radioactive coatings layers. In a particular embodiment of the method, the radioisotope of the first coating layer is ¹⁹⁸Au, while the radioisotope of one or more additional coating layers is ³²P.

In one embodiment of the method of the present invention, an additional protective coating is formed over the radioactive coating or over the top radioactive coating where multiple radioactive coatings present in layers. This protective coating seals the radioactive coating and prevents dissolution of radioisotope in solution due to, for example, corrosion or abrasion. In a particular embodiment, the protective layer may formed by coating a Ni coating solution onto a radioactive coating by, for example, electrodeposition or electroless deposition. The protective layer, unlike the radioactive composite coating, does not contain radioisotope.

The invention disclosed herein also relates to radioactive coated substrates. Radioactive substrates have a variety of industrial and medical applications. It is known, for example, that radiation can be used to inhibit cell proliferation. Thus, radioactive substrates may be useful in treating a variety of diseases associated with aberrant cell proliferation, including cancer and arterial restenosis. One purpose of the present invention, therefore, is to provide radioactive substrates useful in the treatment of human disease. More specifically, a particular purpose of the present invention is to provide radioactive substrates useful in the treatment of cancer and vascular disease.

In one embodiment, the present invention relates to a coated substrate comprising at least a first layer of a radioactive coating disposed on a substrate material, wherein the radioactive coating comprises at least one carrier metal and a radioisotope. The carrier metal and radioisotope can be those carrier metals and radioisotopes identified herein for use in the radioactive coating solutions of the present invention, as would be understood by one skilled in the art. In a particular embodiment, the coating comprises Ni and phosphorus, wherein at least a fraction of the phosphorus is ³²P. The coating may have a P content ranging, for example from low (1-4 weight %P) to medium (5-8 weight %P) to high (9-16 weight %P). In a particular embodiment, the fraction of P that is ³²P is about 0.01% or less. Optionally, a catalytic coating layer or activation layer is also present, interposed between the substrate and the first layer of radioactive coating. For example, a non-radioactive Ni coating may be interposed between the substrate and the first radioactive coating layer.

In a further embodiment, the present invention relates to a coated substrate comprising at least a first layer of a radioactive composite coating comprising a metal matrix and a radioactive phase dispersed therein, disposed over a substrate material. The metal matrix may be formed of those metal identified herein for use in the radioactive coating solutions of the present invention, as would be understood by one skilled in the art. Similarly, the radioactive phase may be formed of those insoluble radioisotopes or insoluble compounds of radioisotopes identified herein for use in the radioactive coating solution of the present invention, as would be understood by one skilled in the art. Optionally, a catalytic coating layer (e.g., a non-radioactive Ni coating) is also present, interposed between the substrate and the first radioactive coating layer.

The present invention is also directed to substrates comprising multiple radioactive coating layers, which coating layers may be the same or different in composition or method of deposition, or both. Optionally, one or more catalytic coating layers may be interposed between one or more of the multiple radioactive coating layers. A activation or catalytic layer may also be formed onto the substrate prior to deposition of a radioactive coating layer thereon. In one embodiment of the present invention, the first layer of radioactive coating is different from at least one or more additional layers. For example, the radioisotope of the first layer is different from the radioisotope of at least one or more additional layers. In a particular embodiment, the radioisotope of one layer of radioactive coating is ¹⁹⁸Au, while the radioisotope of one or more additional layers is ³²P. Multiple radioactive coatings layers may be deposited by electrodeposition or electroless deposition, sol-gel methods, or a combination thereof. Suitable substrates include, but are not limited to, metals, alloys, polymers, plastics, ceramics and composites.

FIG. 33B depicts a substrate comprising multiple radioactive coating layers. A nickel substrate (1) is shown having an electrodeposited Au/¹⁹⁸Au layer (2) formed thereon. An electro deposited Ni activation layer (3) if further formed on top of the Au/¹⁹⁸Au layer (2). Electrolessly deposited onto the Ni activation layer (3) is a Ni—P/³²P coating layer (4). Finally, a protective coating (5) comprising Ni—P is formed by electroless deposition onto the coated substrate.

The present invention also relates to the method of ion implantation as a surface modification technique to render the medical devices according to this invention radioactive. In a particular embodiment of this method, a source of ³²P is generated and accelerated to a voltage of 100 keV. (A range of voltage may be used, between 25 keV and 500 keV, typically between 50 and 150 keV). An unmounted metal mesh suitable for subsequent use in a dilation/perfusion device is placed in the end station of an ion implanter, on a rotatable platform. This platform allows for the rotation of the device to allow for ion implantation to occur on all sides of the device, in order to evenly distribute ³²P over the surface of the device. Grounding of the device is achieved through use of a wire, which also serves to measure the total beam current delivered to the device, to allow a direct measure of beam current. In order to activate a device to a total activity of 20 mCi, approximately 2.2×10⁻⁶ m-mole of ³²P is delivered to the device. This method advantageously provides ³²P that is embedded within approximately the top 1 micron of alloy. Further, the radioisotope is not present as a surface layer, but rather as a surface alloy that is an integral part of the substrate and therefore not subject to delamination.

In a particular embodiment, the substrate of the present invention is a medical device formed from, for example, materials such as metals, alloys, polymers, ceramics or composites, or a combinations thereof. Non-limiting examples of medical devices which are suitable substrates for the present invention include guidewires, stents, brachytherapy devices and catheters with expandable mesh components. More particularly, the substrate of the present invention may be the expandable mesh component of a dilatation/perfusion catheter previously described.

### EXAMPLE 1

100 mls of an electroless nickel coating solution was made using two commercially available electroless nickel-phosphorous concentrates, including 6.5 mls of Niklad 1000A and 15 mls of Niklad 1000B, (both from MacDermit, Inc.), the remainder de-ionized water according to Niklad product specifications. This solution until the total volume was 80 mls. Subsequently, 7.8 mls of the concentrated solution were placed into a 15 ml test-tube behind a shielded hood, and 2.08 mis of radioactive hypophosphite solution ions (custom synthesized by NEN Life Science Products, containing a mixture of PO₂ and PO₃/PO₄ in a ratio of approximately 10:90 was added thereto. The total activity added to the test tube was approximately 25 mCi of ³²P, and thus contained approximately 2.5 mCi of ³²P in the form of hypophosphite ion (H₂PO₂). The solution was heated to approximately 88° C. on a hot place, with the solution agitated by means of a stir bar.

A catheter sample, the FullFlow™ Device, manufactured by InterVentional Technologies, Inc., was inserted into the solution after having been plated with Ni to activate the surface to be coated, and coated for 40 minutes. Hydrogen bubbles that were produced on the sample surface almost immediately on insertion indicated that the sample was being coated. Bubbling appeared uniform over across the entire sample surface, and the bubbling rate appeared constant over the 40 minute coating period. The device was then removed from the coating solution and rinsed thoroughly.

The radioactivity of the sample was determined using a GM detector. The reading from the GM detector, held next to the catheter after it was rinsed, exceeded 300,000 counts using a 4% efficient GM detector. The catheter was also placed into a liquid scintillation vial and assayed, yielding a reading of 1.08 microcuries.

The uniformity of the radioactive coating was characterized by wrapping GAF-chromic film around the catheter for 16 hours. FIG. 1 shows the optical density readings from the film when measured along the catheter's long axis. FIG. 2 shows the optical density readings from the film when measured along the catheter's short axis. Absolute activity is not known, given the absence of a standard. An estimate of the catheter's activity, based upon a 1% yield of hypophosphite in solution to phosphorus on the part, is about 25 μCi. This experiment shows that hypophosphite having at least a portion of P as ³²P when present in an electroless Ni coating solution can indeed cause ³²P-containing Ni—P deposits to be produced. Scale-up of the quantity of ³²P added to the solution described above by a factor of approximately 1000 would cause a substrate or component to be produced having about 25 mCi of activity, which level of activity is desirable for use in applications involving coronary angioplasty for example.

### EXAMPLE 2

A solution was prepared according to the recipe below:

Ni—P bath:

150 g/l NiSO₄ 6 H₂O;

45 g/l NiCl₂ 6 H₂O;

45 g/l NiCO₃;

50 g/l H₃PO₃;

40 g/l H₃PO₄;

saccharin 5 g/l

Approximately 16 mls of solution was placed into a cylindrical plating cell with a circumferential Ni anode. Approximately 50 mCi of ³²P-phosphorous acid (containing some non-radioactive phosphorous acid carrier material) was added to the cell. HCl was added to the cell to bring the pH to 0.83+/−0.04. Distilled water was added to bring the total volume of solution to 20 mls. A 3.0 mm diameter by 20 mm length FullFlow device with appropriate surface pre-treatment (degreasing, deoxidizing, HCl and Ni strike treatments) was then inserted into the plating cell, and electrodeposited with a current density of 0.12 amps for 25 minutes. The solution temperature was maintained at 60° C. and there was continuous bath agitation during electrodeposition. The sample was removed, rinsed copiously and dried.

An adherent, smooth Ni—P coating exhibiting 16 weight percent P was produced, yielding a catheter with a total activity of 200 uCi.

While the foregoing specification teaches the principles of the present invention, with examples provided for the purpose of illustration, it will be understood that the practice of the invention encompasses all of the usual variations, adaptations, and modifications, as come within the scope of the following claims and their equivalents.

We claim:

1. A method for dilating and irradiating a vascular segment or a body passageway which comprises:
exposing an expansion member to an electroless deposition method such that said expansion member becomes radioactive;
said expansion member being moveable between a first radially contracted configuration and a second radially expanded configuration;
placing said radioactive expansion member in its radially contracted configuration into a vascular segment or body passageway;
advancing said radioactive expansion member to a predetermined site in the vascular segment or body passageway in said radially contracted configuration;
altering the configuration of said radioactive expansion member from said radially contracted configuration to said radially expanded configuration wherein said expansion member dilates and irradiates said predetermined site while allowing fluid to flow through said expansion member; and
altering the configuration of said radioactive expansion member from said radially expanded configuration substantially to said radially contracted configuration for removal of said expansion member from said vascular segment or body passageway.

2. The method according to claim 1, wherein said electroless deposition method comprises:
(a) contacting said expansion member with a radioactive coating solution under conditions sufficient to chemically deposit at least one radioactive composite coating layer onto said expansion member; and
(b) removing any excess or spent coating solution from the expansion member, thereby forming a radioactive expansion member,
wherein said coating solution comprises:
(1) at least one dissolved carrier metal ion; and
(2) a reducing agent; and
(3) either an insoluble radioisotope or an insoluble compound of a radioisotope suspended therein.

3. The method according to claim 1, wherein said expansion member has a circumferential dimension and an axial dimension, said expansion member including a radioactive coating that has a total radioactivity that varies in at least one of said circumferential dimension and said axial dimension.

4. A method for dilating and irradiating an obstruction in a vascular segment or a body passageway which comprises:
exposing an expansion member to an electroless deposition method such that said expansion member becomes radioactive;
said expansion member being moveable between a first radially contracted configuration and a second radially expanded configuration;
placing said radioactive expansion member in its radially contracted configuration into a vascular segment or body passageway;
advancing said radioactive expansion member to a predetermined site in the vascular segment or body passageway in said radially contracted configuration;
altering the configuration of said radioactive expansion member from said radially contracted configuration to said radially expanded configuration, wherein said expansion member dilates and irradiates said obstruction at said predetermined site while allowing fluid to flow through said expansion member; and
altering the configuration of said radioactive expansion member from said radially expanded configuration substantially to said radially contracted configuration for removal of said expansion member from said vascular segment or body passageway.

5. The method according to claim 4, wherein said electroless deposition method comprises:
(a) contacting said expansion member with a radioactive coating solution under conditions sufficient to chemically deposit at least one radioactive composite coating layer onto said expansion member; and
(b) removing any excess or spent coating solution from the expansion member, thereby forming a radioactive expansion member,
wherein said coating solution comprises:
(1) at least one dissolved carrier metal ion; and
(2) a reducing agent; and
(3) either an insoluble radioisotope or an insoluble compound of a radioisotope suspended therein.

6. A method for dilating and irradiating a vascular segment or a body passageway which comprises:
exposing an expansion member to an electrodeposition method such that said expansion member becomes radioactive;
said expansion member being moveable between a first radially contracted configuration and a second radially expanded configuration;
placing said radioactive expansion member in its radially contracted configuration into a vascular segment or body passageway;
advancing said radioactive expansion member to a predetermined site in the vascular segment or body passageway in said radially contracted configuration;
altering the configuration of said radioactive expansion member from said radially contracted configuration to said radially expanded configuration, wherein said expansion member dilates and irradiates said predetermined site while allowing fluid to flow through said expansion member; and
altering the configuration of said radioactive expansion member from said radially expanded configuration substantially to said radially contracted configuration for removal of said expansion member from said vascular segment or body passageway.

7. The method according to claim 6, wherein said electrodeposition method comprises:
(a) contacting the expansion member with a radioactive coating solution under conditions sufficient to electrically deposit at least one radioactive composite coating layer onto the expansion member; and
(b) removing any excess or spent coating solution from the expansion member, thereby forming a substrate comprising a radioactive composite coating,
wherein said coating solution comprises:
(1) at least one dissolved carrier metal ion; and
(2) either an insoluble radioisotope or an insoluble compound of a radioisotope.

8. The method of claim 7, wherein the radioactive expansion member emits beta radiation.

9. The method of claim 8, wherein the radioactive expansion member comprises a non-metallic radioactive coating.

10. A method for dilating and irradiating an obstruction in a vascular segment or a body passageway which comprises:
exposing an expansion member to an electrodeposition method such that said expansion member becomes radioactive;
said expansion member being moveable between a first radially contracted configuration and a second radially expanded configuration;
placing said radioactive expansion member in its radially contracted configuration into a vascular segment or body passageway;
advancing said radioactive expansion member to a predetermined site in the vascular segment or body passageway in said radially contracted configuration;
altering the configuration of said radioactive expansion member from said radially contracted configuration to said radially expanded configuration, wherein said expansion member dilates and irradiates said obstruction at said predetermined site while allowing fluid to flow through said expansion member; and
altering the configuration of said radioactive expansion member from said radially expanded configuration substantially to said radially contracted configuration for removal of said expansion member from said vascular segment or body passageway.

11. The method according to claim 10, wherein said electrodeposition method comprises:
(a) contacting the expansion member with a radioactive coating solution under conditions sufficient to electrically deposit at least one radioactive composite coating layer onto the expansion member; and
(b) removing any excess or spent coating solution from the expansion member, thereby forming a substrate comprising a radioactive composite coating, wherein said coating solution comprises:
(1) at least one dissolved carrier metal ion; and
(2) either an insoluble radioisotope or an insoluble compound of a radioisotope.

12. The method according to claim 10, wherein said expansion member has a circumferential dimension and an axial dimension, said expansion member including a radioactive coating that has a total radioactivity that varies in at least one dimension of the expansion member.

13. The method of claim 10, wherein the radioactive expansion member emits beta radiation.

14. The method of claim 13, wherein the radioactive expansion member comprises a non-metallic radioactive coating.

15. A catheter for dilating and irradiating a vascular segment or a body passageway which comprises:
a distal end and a proximal end;
a substantially cylindrical shaped expansion member located on said distal end of said catheter, said expansion member having a first end and a second end, said first end being a distance from said second end;
an altering mechanism engagable to said first end and said second end of said expansion member for altering said first distance therebetween to move said expansion member between a first configuration wherein said expansion member is characterized by a first diameter and a second configuration wherein said expansion member is characterized by a second diameter, said second diameter being greater than said first diameter; and
a radioactive source located at said distal end of said catheter,
wherein said radioactive source includes a radioactive coating formed by an electroless deposition method.

16. The catheter according to claim 15, wherein said electroless deposition comprises:
(a) contacting the catheter with a radioactive coating solution under conditions sufficient to chemically deposit at least one radioactive composite coating layer onto the catheter; and
(b) removing any excess or spent coating solution from the catheter, thereby forming a catheter comprising a radioactive composite coating, wherein said coating solution comprises:
(1) at least one dissolved carrier metal ion;
(2) a reducing agent; and
(3) either an insoluble radioisotope or an insoluble compound of a radioisotope suspended therein.

17. The catheter according to claim 15, wherein said radioactive source is said substantially cylindrical shaped expansion member.

18. A mechanical dilatation and irradiation device comprising:
a catheter having a distal end and a proximal end, said catheter having an inner member and an outer member;
an expandable mesh positioned on said distal end adapted to dilate an obstruction in a vascular segment, said mesh having a first contracted diameter and a second expanded diameter, said second expanded diameter being larger than said first contracted diameter;
said device being adapted to dilate said obstruction and expose said obstruction to radiation; and
said device having a radioactive source located at said distal end,
wherein said radioactive source includes a radioactive coating formed by electroless deposition.

19. The mechanical dilatation and irradiation device according to claim 18, wherein said electroless deposition comprises:
(a) contacting the device to be coated with a radioactive coating solution under conditions sufficient to chemically deposit at least one radioactive composite coating layers onto the device; and
(b) removing any excess or spent coating solution from the device, thereby forming a mechanical dilatation and irradiation device comprising a radioactive composite coating,
wherein said coating solution comprises:
(1) at least one dissolved carrier metal ion;
(2) a reducing agent; and
(3) either an insoluble radioisotope or an insoluble compound of a radioisotope suspended therein.

20. The mechanical dilatation and irradiation device according to claim 18, wherein said radioactive source is said expandable mesh.

21. A catheter for dilating and irradiating an obstruction within a vascular segment or a body passageway which comprises:
a distal end and a proximal end;
a substantially cylindrical shaped expansion member located on said distal end of said catheter, said expansion member having a first end and a second end, said first end being a distance from said second end;
an altering mechanism engagable to said first end and said second end of said expansion member for altering said first distance therebetween to move said expansion member between a first configuration wherein said expansion member is characterized by a first diameter and a second configuration wherein said expansion member is characterized by a second diameter, said second diameter being greater than said first diameter; and
a radioactive source located at said distal end of said catheter,
wherein said radioactive source includes a radioactive coating formed by electrodeposition.

22. The catheter according to claim 21, wherein said electrodeposition comprises:
(a) contacting the catheter with a radioactive coating solution under conditions sufficient to electrically deposit at least one radioactive composite coating layers onto the catheter; and
(b) removing any excess or spent coating solution from the catheter, thereby forming a catheter having a radioactive composite coating, wherein said coating solution comprises:
(1) at least one dissolved carrier metal ion; and
(2) either an insoluble radioisotope or an insoluble compound of a radioisotope.

23. The catheter according to claim 21, wherein said radioactive source is said substantially cylindrical expansion member.

24. The catheter of claim 21, wherein the radioactive expansion member emits beta radiation.

25. The catheter of claim 24, wherein the radioactive expansion member comprises a non-metallic radioactive coating.

26. A mechanical dilatation and irradiation device comprising:
a catheter having a distal end and a proximal end, said catheter having an inner member and an outer member;
an expandable mesh positioned on said distal end adapted to dilate an obstruction in a vascular segment, said mesh having a first contracted diameter and a second expanded diameter, said second expanded diameter being larger than said first contracted diameter;
said mechanical dilatation and irradiation device being adapted to dilate said obstruction and expose said obstruction to radiation; and
said mechanical dilatation and irradiation device having a radioactive source located at said distal end,
wherein said radioactive source includes a radioactive coating formed by electrodeposition.

27. The mechanical dilatation and irradiation device according to claim 26, wherein said electrodeposition comprises:
(a) contacting the catheter with a radioactive coating solution under conditions sufficient to electrically deposit at least one radioactive composite coating layers onto the catheter; and
(b) removing any excess or spent coating solution from the catheter, thereby forming a catheter comprising a radioactive composite coating,
wherein said coating solution comprises:
(1) at least one dissolved carrier metal ion; and
(2) either an insoluble radioisotope or an insoluble compound of a radioisotope.

28. The mechanical dilatation and irradiation device according to claim 26, wherein the radioactive expansion member emits beta radiation.

29. The mechanical dilatation and irradiation device according to claim 28, wherein the radioactive expansion member comprises a non-metallic radioactive coating.

30. An assembly comprising:
a catheter for dilating and irradiating a vascular segment or a body passageway; and a stent located over said catheter, said assembly comprising:
a distal end and a proximal end;
a substantially cylindrical shaped expansion member located on said distal end of said assembly, said expansion member having a first end and a second end, said first end being a distance from said second end;
an altering mechanism engagable to said first end and said second end of said expansion member for altering said first distance therebetween to move said expansion member between a first configuration wherein said expansion member is characterized by a first diameter and a second configuration wherein said expansion member is characterized by a second diameter, said second diameter being greater than said first diameter, wherein said expansion member radially expands said stent when said expansion member is in said second diameter; and
a radioactive source located at said distal end of said assembly,
wherein said radioactive source includes a radioactive layer formed by electroless deposition, electodeposition or ion implantation.

31. The assembly according to claim 30, wherein the radioactive source has a circumferential dimension and an axial dimension, said radioactive coating has a total radioactivity that varies in at least one of said circumferential dimension and said axial dimension.

32. A catheter for dilating and irradiating a vascular segment or a body passageway which comprises:
a distal end and a proximal end;
a substantially cylindrical shaped expansion member located on said distal end of said catheter, said expansion member having a first end and a second end, said first end being a distance from said second end;
an altering mechanism engagable to said first end and said second end of said expansion member for altering said first distance therebetween to move said expansion member between a first configuration wherein said expansion member is characterized by a first diameter and a second configuration wherein said expansion member is characterized by a second diameter, said second diameter being greater than said first diameter; and
a radioactive source located at said distal end of said catheter,
wherein said radioactive source includes a radioactive layer formed by ion implantation.

33. A method for dilating and irradiating a vascular segment or body passageway or obstruction in said vascular segment or body passageway, said method comprises:
exposing an expansion member to ion implantation such that said expansion member becomes radioactive;
said expansion member being moveable between a first radially contracted configuration and a second radially expanded configuration;
placing said radioactive expansion member in its radially contracted configuration into a vascular segment or body passageway;
advancing said radioactive expansion member to a predetermined site in the vascular segment or body passageway in said radially contracted configuration;
altering the configuration of said radioactive expansion member from said radially contracted configuration to said radially expanded configuration wherein said expansion member dilates and irradiates said predetermined site while allowing fluid to flow through said expansion member; and
altering the configuration of said radioactive expansion member from said radially expanded configuration substantially to said radially contracted configuration for removal of said expansion member from said vascular segment or body passageway.

34. The method of claim 33, wherein the radioactive expansion member emits beta radiation.

35. The method of claim 34, wherein the radioactive expansion member comprises a non-metallic radioactive coating.

36. The method according to claim 33, wherein said expansion member has a circumferential dimension and an axial dimension, said expansion member including a radioactive coating that has a total radioactivity that varies in at least one of said circumferential dimension and said axial dimension.
